SEQUENCE LISTING (6 sequences)

SEQ ID NO: 1 - DNA, 780 bp, Yersinia pestis
  CDS (1)..(777)
atcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 2 - protein, 259 aa, Yersinia pestis
IELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 3 - DNA, 513 bp, Yersinia pestis
  CDS (1)..(510)
atgaaaaaaatcagttccgttatcgccattgcattatttggaactattgcaactgctaatgcggcagatttaactgcaagcaccactgcaacggcaactcttgttgaaccagcccgcatcactcttacatataaggaaggcgctccaattacaattatggacaatggaaacatcgatacagaattacttgttggtacgcttactcttggcggctataaaacaggaaccactagcacatctgttaactttacagatgccgcgggtgatcccatgtacttaacatttacttctcaggatggaaataaccaccaattcactacaaaagtgattggcaaggattctagagattttgatatctctcctaaggtaaacggtgagaaccttgtgggggatgacgtcgtcttggctacgggcagccaggatttctttgttcgctcaattggttccaaaggcggtaaacttgcagcaggtaaatacactgatgctgtaaccgtaaccgtatctaaccaataa

SEQ ID NO: 4 - protein, 170 aa, Yersinia pestis
MKKISSVIAIALFGTIATANAADLTASTTATATLVEPARITLTYKEGAPITIMDNGNIDTELLVGTLTLGGYKTGTTSTSVNFTDAAGDPMYLTFTSQDGNNHQFTTKVIGKDSRDFDISPKVNGENLVGDDVVLATGSQDFFVRSIGSKGGKLAAGKYTDAVTVTVSNQ

SEQ ID NO: 5 - DNA, 264 bp, Yersinia pestis
  CDS (1)..(261)
atgagtaacttctctggatttacgaaaggaaccgatatcgcagacttagatgcggtggctcaaacgctcaagaagccagcagacgatgcaaacaaagcggttaatgactcgatagcagcattgaaagataagcctgacaacccggcgctacttgctgacttacaacattcaattaataaatggtcggtaatttacaatataaactcaaccatagttcgtagcatgaaagacttaatgcaaggcatcctacagaagttcccataa

SEQ ID NO: 6 - protein, 87 aa, Yersinia pestis
MSNFSGFTKGTDIADLDAVAQTLKKPADDANKAVNDSIAALKDKPDNPALLADLQHSINKWSVIYNINSTIVRSMKDLMQGILQKFP

(19) United States
(12) Patent Application Publication
(10) Pub. No.: US 2010/0209451 A1
(43) Pub. Date: Aug. 19, 2010

(54) Compositions and Methods Related to Adenovirus Based Delivery of Antigens

(75) Inventor: Peter Clarke, Sugar Land, TX (US)

Correspondence Address: FULBRIGHT & JAWORSKI L.L.P., 600 CONGRESS AVE., SUITE 2400, AUSTIN, TX, 78701, US

(73) Assignee: INTROGEN THERAPEUTICS, INC.

(21) Appl. No.: 11/764,719

(22) Filed: Jun. 18, 2007

Related U.S. Application Data
(60) Provisional application No. 60/814,281, filed on Jun. 16, 2006.

(51) Int. Cl. A61K 39/12; C12N 15/74; C07H 21/04; A61K 39/23; A61P 31/12; A61P 37/04

(52) U.S. Cl. 424/199.1; 435/320.1; 536/23.72; 424/233.1

(57) ABSTRACT

Embodiments of the present invention include the construction and generation of a multi-use adenoviral vaccine platform applicable to biodefense, and emerging and re-emerging infectious diseases. Adenoviral vaccines of the invention will elicit an immune response against pathogenic organisms that cause such infectious diseases. In certain aspects of the invention, the pathogenic organisms include, but are not limited to EEEV and Y. pestis. Further embodiments of the invention include compositions and methods related to such adenoviral vaccines.

This application claims priority to U.S. provisional patent application Ser. No. 60/814,281 filed on Jun. 16, 2006, which is hereby incorporated by reference.

The United States Government may own rights in the present invention pursuant to grant [pending] from the National Institute of Allergy and Infectious Disease.

### BACKGROUND OF THE INVENTION

I. Field of the Invention

This invention relates to the fields of immunology, microbiology, medicine, and infectious diseases. Particular aspects of the invention relate to methods and compositions that prevent, treat, or attenuate the pathological effects of exposure to one or more pathogenic organism or toxin, particularly biological warfare agents.

II. BACKGROUND

Biological warfare can be used to decimate human populations and to destroy livestock and crops of economic significance. Terrorist attacks in the U.S. and elsewhere have brought into focus the threat posed by biological weapons and have provoked discussion of mass vaccination strategies for both military personnel and civilian populations. The strategies assume the use of classical bioweapons agents. However, the power of genetic engineering raises the possibility of an advanced generation of bioweapons agents that are even more virulent than their naturally occurring counterparts and that are capable of evading current vaccine defenses.

The list of classical biological agents that could be used as bioweapons includes over 100 bacteria, viruses, rickettsia, fungi, and toxins. However, most experts believe that the most likely bioweapons include anthrax, smallpox, plague, botulinum toxin, tularemia, and viral hemorrhagic fevers. Bioengineering of these materials to create artificial viruses, antibiotic resistant strains of microorganisms, toxins and other exotic bioweapons is a distinct possibility.

In some cases an alphavirus (e.g., Eastern Equine Encephalitis Virus (EEEV)) or the bacterium Yersinia pestis may be used as such a weapon. Currently there are no commercially available vaccines in the U.S. for human use to protect against infection with EEEV or Y. pestis.

Eastern Equine Encephalitis Virus. EEEV, like its sister pathogens Venezuelan and Western equine encephalitis viruses, is a highly developed biological warfare agent as well as a naturally occurring and emerging zoonotic virus. The lack of a vaccine is particularly worrisome because of the pathogen's capability to inflict widespread morbidity and mortality upon exposure to both civilians and military personnel. Both the United States and the Soviet Union had active programs for the development of EEEV weapons. The U.S. program was halted in 1969, but the Soviets' development of the weapon proceeded until the mid-1990s. Further, the recruitment of Soviet trained scientists by rogue nations has increased the possibility of this threat. Even with the high political costs associated with state-sponsored terrorism, the threat still remains for both terrorism and criminally motivated attacks. And because the virus is a naturally occurring zoonotic pathogen, its availability and a rapidly shrinking technological barrier would make it an attractive target for aspiring perpetrators.

Other causes for concern include: (a) aerosolization technology for infectious material is highly developed in the U.S. and former Soviet Union; (b) the virus generates little natural immunity in human populations; (c) the virus replicates to very high titer in a variety of cell cultures (10⁹-10¹⁰ infectious units/ml); (d) the virus is highly stable when lyophilized, and is “user friendly” compared to many other viral BWT agents; (e) is infectious by aerosol, with several documented laboratory aerosol infections (U.S. Department of Health and Human Services, 1999); and (f) it has the highest rate of neurological disease among apparent alphavirus infections, with mortality rates of about 30-70%.

Current experimental vaccines include those produced decades ago by the U.S. military. These vaccines include formalin-inactivated formulations prepared from a wild-type, virulent strain that typically require 3 injections to induce protective immunity and frequent boosters for maintenance of neutralizing antibodies. The risk of administering formalin-inactivated vaccines prepared from virulent, wild-type alphavirus strains was underscored by Venezuelan equine encephalitis (VEE) epidemics (Weaver et al., 1999) and a recent equine EEE case (Franklin et al., 2002) that were probably initiated by residual live virus. No effective antivirals have been developed against EEEV, making vaccines the only line of defense (Tsai et al., 2002).

Yersinia pestis. Y. pestis is a Gram-negative bacterium that is the etiological agent of bubonic and pneumonic plague. The plague is a zoonotic infection and can be transmitted to humans via a bite from a flea that previously fed on an infected rodent. Typically, flea transmission of Y. pestis causes a form of disease referred to as bubonic plague. From the initial site of infection, bacteria disseminate to the draining lymph node, causing the lymph node to form a bubo (an inflammatory swelling of a lymph gland). Infection can spread from such a bubo into the circulation, eventually causing bacteremia and the second form of the disease, septicemic plague. Sometimes septicemic disease occurs even without the development of buboes and is characterized by an elevated temperature, chills, headache, malaise and gastrointestinal disturbances. In addition, if the lungs become infected, pneumonic plague can result. Pneumonic plague is the most feared form of the disease that arises due to colonization of the alveolar spaces, and can also be caused by bacterial spread from an infected person (or animal) to a healthy individual by the aerosol route. Pneumonic plague develops rapidly (1-3 days), results in high mortality in infected individuals (approaching 100%), and spreads rapidly from human-to-human. Y. pestis has been responsible for at least three pandemics in the past, killing by estimation more than 200 million people (Perry and Fetherston, 1997). For that reason, and because plague is characterized as an emerging infectious disease, the Centers for Disease Control and Prevention has classified it as a category A biological agent.

The vaccine previously licensed in the U.S. was a formaldehyde-killed preparation of the highly virulent 195/P strain of Y. pestis. This vaccine required a course of injections over a period of 6 months and was effective against bubonic plague. However, the protection was short-term and annual boosters were required; the incidence of side effects, such as malaise, headaches, elevated temperature and lymphadenopathy, was high (in approximately 10% of those immunized with vaccines); and the vaccine was expensive. Moreover, the protection of killed, whole-cell vaccine against the pneumonic form of plague was uncertain. A live attenuated vaccine (Y. pestis pigmentation-negative mutant EV76) is also available. This type of vaccine has been used for almost a century and has proven effective against subcutaneous and inhalation challenges with Y. pestis. However, the safety of this vaccine in humans is questionable, because it retains some virulence, and in most countries (including the U.S.) live vaccines such as this are not licensed.

Thus, there remains a need for additional materials and methods for protection and treatment against a variety of pathogenic organisms or toxins, particularly EEEV and Y. pestis.

### SUMMARY OF THE INVENTION

Vaccination has not only been one of the most significant advancements in healthcare, but also a cost-effective means of public health intervention. While conventional vaccine strategies have focused on live-attenuated or killed virus approaches, a new approach to the development of vaccines utilizes platform technologies and scientific advancement to overcome the challenges in vaccine design. Embodiments of the present invention include the construction and generation of a multi-use adenoviral vaccine platform applicable to biodefense, and emerging and re-emerging infectious diseases. Adenoviral vaccines of the invention will elicit an immune response against pathogenic organisms that cause such infectious diseases or toxins that result in a pathological response in a subject. In certain aspects of the invention, a pathogenic organism includes, but is not limited to EEEV and Y. pestis.

This invention may be used in the vaccination of individuals exposed or at risk of exposure to one or more infective or pathogenic agents, such as a pathogenic organism or a toxin. A number of pathogenic organisms are potential biowarfare agents. Embodiments of the invention include methods and compositions for vaccines expressing antigens from one or more biothreat agents to provide protection against or therapy for an array of infective organisms, such as bioweapons, including, but not limited to pathogenic bacteria, fungi, and/or viruses. These pathogenic organisms may be isolated from nature or engineered or selected for a pathogenic phenotype.

As used herein, the term “antigen” is a molecule capable of being bound by an antibody or T-cell receptor. An antigen is additionally capable of inducing a humoral immune response and/or cellular immune response leading to the production of B- and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another.

Aspects of the invention include recombinant adenovirus vectors comprising one or more heterologous DNA segment(s) encoding one or more antigenic determinants from one or more target organisms that elicit an immune response to one or more pathogenic organism, a component of a pathologic organism, or a toxin. In certain aspects the pathogenic organism or toxin is a biological weapon. The pathogenic organisms can be an organism classified as a category A, category B, or category C organism. Further aspects of the invention include fungal, viral, or bacterial organisms. In a particular aspect the organism is a virus (e.g., EEEV) or a bacterium (e.g., Y. pestis). A virus can be an alphavirus, more particularly an equine encephalitis virus. Embodiments of the invention include adenoviral vaccines comprising a DNA segment encoding an antigenic determinant of an eastern equine encephalitis virus (EEEV). The antigenic determinant can include all or part of an E3, an E2, a 6K, and/or an E1 region of EEEV, or any combination thereof. Aspects of the invention include an adenoviral vaccine encoding E3, E2, 6 k, and E1 regions of EEEV. Other aspects of the invention include adenoviral vaccines encoding at least the E3-E2 regions of EEEV. A heterologous DNA segment is typically included in an expression cassette.

In another embodiment of the invention an adenoviral vaccine can include an antigenic determinant of a bacteria. In particular embodiments the bacteria is Yersinia, more particularly Y. pestis. The antigenic determinant of Yersinia can include all or part of a Caf1, a LcrV, a YscF proteins, or any combination thereof. In certain aspects, adenoviral vaccine includes an antigenic determinant comprising all or part of a Caf1 protein, a LcrV protein, and/or a YscF protein. Embodiments of the invention also include an adenoviral vaccine including an antigenic determinant having all or part of a Caf1 protein, all or part a LcrV protein, and all or part of a YscF protein.

Further embodiments of the invention include isolated nucleic acids comprising an adenoviral genome comprising a heterologous DNA segment encoding an antigenic determinant of a category A, category B, or category C organism, as classified by the United States Centers for Disease Control.

Still further embodiments include an immunogenic composition comprising an adenovirus encoding an antigenic determinant of a category A, category B, or category C organism.

Methods of the invention include therapeutic and/or prophylactic immunization of a subject comprising administering a recombinant adenovirus encoding an antigenic determinant of a category A, category B, or category C organism.

Embodiments of the invention also include methods for producing or inducing a protective immune response against a category A, category B, and/or category C organism, particularly those organisms that are capable of being weaponized, in a mammal. Methods of the invention comprise administering to such a mammal a vaccine comprising an adenoviral vector having a heterologous DNA segment encoding an antigenic determinant of the category A, category B, and/or category C organism. A subject may include, but are not limited to humans, horses, cows, sheep, goats, fowl, chickens, dogs, cats, rats, mice, pigs and the like.

Other aspects of the invention include methods for readily producing large quantities of an adenoviral vaccine that are stable at room temperature, at refrigerated temperature or temperatures below freezing over time.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

FIG. 1. Genome organization of alphaviruses. The nonstructural protein genes (nsP1-4) are located at the 5′ end of the genome. The 26S subgenomic message is identical to the 3′ one third of the genome and encodes the structural proteins [capsid (C) and E2 and E1 envelope glycoproteins].

FIGS. 2A-2C Infection of NIH Swiss mice (FIG. 2A), Vero (FIG. 2B) and C6136 mosquito (FIG. 2C) cells with EEEV strain FL93-939 or virus derived from the infectious cDNA clone derived from this strain, see Wang et al., 2006, which is incorporated herein by reference in its entirety. Animals received a sc dose of 10³ PFU, and cell cultures were infected with a multiplicity of 0.1. After adsorption of virus to cell cultures, they were washed 3 times with PBS, then cell culture medium added and sampled to determine the residual, time-0 titers. Bars indicate standard errors.

FIG. 3. Silver stained gel containing purified recombinant antigens of Y. pestis CO92. YopM and E represent T3SS effectors and Pla is a plasminogen activator protease.

FIGS. 4A-4D. Characteristic histopathological changes in liver caused by Y. pestis KIM on postinfection day 3. (FIG. 4A) Lcr+ Y. pestis cells in mouse immunized with PA, showing multiple focal necrotic lesions without inflammation; (FIG. 4B) Lcr-Y. pestis cells in nonimmunized control mouse, exhibiting granuloma formation; (FIG. 4C) Lcr+ Y. pestis cells in mouse actively immunized with PAV, showing protective granulomatous lesions; (FIG. 4D) Lcr+ Y. pestis cells in mouse passively immunized with rabbit anti-PAV, showing lesions prompting accumulation of mononuclear cells.

FIG. 5. Survival of Swiss-Webster mice challenged intranasally with various doses of Y. pestis CO92.

FIG. 6. Survival of Swiss-Webster mice challenged intranasally with various doses of Y. pestis CO92.

FIG. 7. Nucleotide and amino acid sequence homologies between lipoproteins of Y. pestis and Staphylococcus typhimurium.

FIG. 8. Lpp mutant of Y. pestis KIM is attenuated in killing mice.

FIG. 9. Lpp mutant of Y. pseudotuberculosis is attenuated in a mouse model.

### DETAILED DESCRIPTION OF THE INVENTION

Embodiments of the invention include methods and compositions related to adenoviral vaccines that elicit an immune response against various organisms or proteins produced by such organisms. Certain embodiments include vaccines against organisms that may be used as biological weapons, such as Eastern Equine Encephalitis Virus (EEEV) and Yersinia pestis. The present invention includes methods and compositions for the development, production, and evaluation of a vaccine. Aspects of the invention include antigenic peptide sequences encoded in an adenoviral vector system. Adenoviral based vaccines can be constructed and tested against a variety of organisms, including, category A (e.g., Y. pestis), category B (e.g., Eastern Equine Encephalitis Virus (EEEV)) or category C priority organisms.

### I. Vaccine Candidates

In particular aspects of the invention the compositions and methods of the invention may be used to prevent, reduce the risk of or treat infection or exposure to a biological weapon, including intentional exposure of a subject(s) to an infectious agents. These infectious agents comprise gram-positive, gram-negative, intracellular, and extracellular bacteria, as well as a variety of viruses and fungi. The list of classical biological agents that could be used as bioweapons includes over 100 bacteria, viruses, rickettsia, fungi, and toxins. However, most experts believe that the most likely bioweapons include anthrax, smallpox, plague, botulinum toxin, tularemia, and viral hemorrhagic fevers. Using bioengineering of these materials, artificial viruses, antibiotic resistant strains of microorganisms, toxins and other exotic bioweapons can be created.

Category A Diseases/Agents (class A)—The U.S. public health system and primary healthcare providers must be prepared to address various biological agents, including pathogens that are rarely seen in the United States. High-priority agents include organisms that pose a risk to national security because they can be easily disseminated or transmitted from person to person; result in high mortality rates and have the potential for major public health impact; might cause public panic and social disruption; and require special action for public health preparedness. Category A agents include Anthrax (Bacillus anthracis), Botulism (Clostridium botulinum toxin), Plague (Yersinia pestis), Smallpox (variola major), Tularemia (Francisella tularensis), Viral hemorrhagic fevers (filoviruses (e.g., Ebola, Marburg) and arenaviruses (e.g., Lassa, Machupo)).

Category B Diseases/Agents (class B)—Second highest priority agents include those that are moderately easy to disseminate; result in moderate morbidity rates and low mortality rates; and require specific enhancements of CDC's diagnostic capacity and enhanced disease surveillance. Category B agents include Brucellosis (Brucella species), Epsilon toxin of Clostridium perfringens, Food safety threats (e.g., Salmonella species, Escherichia coli O157:H7, Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Viral encephalitis (alphaviruses, e.g., Venezuelan equine encephalitis, eastern equine encephalitis, western equine encephalitis), water safety threats (e.g., Vibrio cholerae, Cryptosporidium parvum).

Category C Diseases/Agents (class C)—Third highest priority agents include emerging pathogens that could be engineered for mass dissemination in the future because of availability; ease of production and dissemination; and potential for high morbidity and mortality rates and major health impact. Category C includes emerging infectious diseases such as Nipah virus and hantavirus.

A. Pathogenic Bacteria

There are numerous bacterial species that are considered pathogenic or potentially pathogenic under certain conditions. These bacteria include, but are not limited to various species of the genus Bacillus, Yersinia, Franscisella, Streptococcus, Staphylococcus, Pseudomonas, Mycobacterium, and Burkholderia. Exemplary bacterial agents include, but are not limited to Bacillus anthracis, Yersinia pestis, Francisella tularensis, Streptococcus pnemoniae, Staphylococcus aureas, Pseudomonas aeruginosa, Burkholderia cepacia, Corynebacterium diphtheriae, Clostridia spp. (Clostridium botulinin, Clostridium perfringens), Shigella spp., Mycobacterium spp, Brucella abortis, B. milletensis, B. suis, and Burkholderia mallei. Embodiments of the invention include compositions and methods that use bacterial antigens in combination with other antigenic/immunogenic proteins or peptides of the same or different bacteria or pathogenic organism, to formulate multi-valent vaccines against one or more organisms or proteins secreted by such, including, but not limited to potential biological weapon.

The genus Yersinia includes Y. pestis, which is a Gram-negative bacterium that is the etiological agent of bubonic and pneumonic plague. Although plague is a zoonotic infection, it could be transmitted to humans via a bite from a flea that previously fed on an infected rodent. The Centers for Disease Control and Prevention has classified Y. pestis as a category A biological agent.

The genus includes three species that are pathogenic for humans, including Y. pestis, and the enteropathogens Y. pseudotuberculosis and Y. enterocolitica. All three species share an almost identical 70-kb virulence plasmid that encodes the type III secretion system (T3SS) essential for basic virulence (Cornelis and Van Gijsegem, 2000). Due to the presence of T3SS, which impairs the innate immune response, in general, and, in particular, affects phagocytosis, Yersinia in visceral organs grows predominantly extracellularly (Brubaker, 2003), and thus Y. pestis occurs within enclosed non-vascularized necrotic foci. These necrotic lesions progressively enlarge and then coalesce as the infection continues (Une et al., 1986). In contrast to enteropathogenic yersiniae that typically harbor a single virulence plasmid, Y. pestis usually possesses two extra plasmids: the large pMT1 that contains genes for capsular antigen F1 (Caf1) and murine toxin; and the small plasmid, pPCP1, encoding the plasminogen activator and bacteriocin pesticin (Perry and Fetherson, 1997).

Vaccines against plague currently under development fall into two major categories. The first is a sub-unit vaccine based on selected plague virulence factors that are highly purified in a recombinant system and used for immunization (ideally as a single-dose delivery). The second is comprised of live attenuated mutants of various pathogens (such as Salmonella, Shigella, Aeromonas, etc.) that carry Y. pestis protective antigens. Also, there are efforts to create a set of controlled mutations in the plague microbe itself to achieve a suitable level of attenuation. Although both subunit and live attenuated vaccines provide a reasonable level of protection against challenge with Y. pestis, there are only two proven protective antigens found thus far, namely Caf1 and LcrV antigens (Titball and Williamson, 2001). Caf1 is a capsular protein, located on the surface of Y. pestis, which is thought to have anti-phagocytic properties (Baker et al., 1952). This capsular antigen has a high molecular weight, polymeric structure and is unique to Y. pestis. The V antigen (LcrV) is a protein that the Y. pestis cell secretes into culture media under certain in vitro growth conditions. LcrV is involved in at least two activities: (i) it participates in the delivery of other Yersinia virulence factors, termed Yops (Yersinia outer membrane proteins), into the host cell (Cornelis, 2002) and (ii) it directs anti-host functions (Brubaker, 2003). LcrV is also present in enteropathogenic Yersinia, and immunization of mice with V antigen derived from Y. pseudotuberculosis has provided strong cross-protection against challenge with plague pathogen (Motin et al., 1994; Nakajima et al., 1995; Motin et al., 1996).

The Caf1 and LcrV antigens have been produced in recombinant form and are used in protection studies, either as a mixture of two proteins or a chimeric F1-V fusion peptide. These antigens have been extensively used in Salmonella based, orally delivered vaccines as well (Oyston et al., 1995; Leary et al., 1997; Garmony et al., 2003). Both proteins were effective in combination and had little competition in eliciting the immune response. The antibody F1+V titers (particularly of the IgG1 isotype) significantly correlated with protection against challenge with plague microbe, including aerosolized Y. pestis (Williamson et al., 1999), and the mechanism of protection also involved T-cell memory, as judged by experiments with IL-4 receptor knock-out mice (Elvin and Williamson, 2000).

Other Y. pestis antigens studied for their efficacy against plague infection did not provide significant protection in a murine model of bubonic plague. Among tested recombinant antigens were T3SS components YpkA, YopH, YopE, YopK, YopN, as well as subunit of pH 6 antigen adhesin and purified LPS. The only protection against mortality was observed in mice vaccinated with YopD, a protein involved in delivery of T3SS effectors into the host cell (Andrews et al., 1999). YopD was cloned and expressed in E. coli as a fusion with polyhistidine tag. The recombinant YopD was purified in its denatured form using 6M urea. Mice immunized with this preparation of YopD were partially protected against approximately 200 LD₅₀; however, YopD-vaccination provided protection only against nonencapsulated version, but not against the capsule Caf1-expressing strain of Y. pestis CO92. The failure of YopD to protect against encapsulated wild-type organisms significantly reduced the value of this antigen as a vaccine candidate.

Recently it was shown that type III secretory needle structure is composed essentially of polymer of YscF subunits, with each subunit having a molecular mass of 6 kDa. The electron microscopy revealed that the isolated needles of Yersinia T3SS were 60-80 nm long and 6-7 nm wide and contained a hollow center of about 2 nm (Hoiczyk and Blobel, 2001). It has been suggested that the needle protein YscF could be an additional antigen for the subunit plague vaccine, since immunization of mice with this protein provided significant protection against challenge with Y. pestis (Matson et al., 2005; Swientnicki et al., 2005). These studies were performed by two independent groups that used similarly constructed recombinant YscF. Vaccination of mice with YscF provided protection against subcutaneous injection of fully virulent encapsulated strain CO92 and against attenuated pigment-negative strain KIM injected intravenously. Although the degree of protection observed after immunization with YscF was less in comparison with that seen for the two known protective antigens, LcrV and Caf1, the protective antigen YscF could be used in combination with LcrV and Caf1 to formulate a tri-valent vaccine for Y. pestis.

Embodiments of the invention include compositions and methods that use YscF antigen in combination with other Yersinia proteins or peptides thereof, such as LcrV and Caf1 antigens to formulate multi-valent vaccines for Y. pestis.

B. Pathogenic Virus

There are numerous viruses and viral strains that are considered pathogenic. Exemplary viral agents include, but are not limited to various strains of alphavirus, hemorrhagic virus, smallpox and others. Particular virus from which a subject may be protected include, but is not limited to alphavirus such as EEEV. Embodiments of the invention include compositions and methods that use viral antigens in combination with other antigenic/immunogenic proteins or peptides of the same or different virus or pathogenic organism, to formulate multi-valent vaccines against one or more organism, including, but not limited to potential biological weapons.

The phrases “structural protein” or “alphavirus structural protein” as used herein refer to one or more of the alphaviral-encoded proteins which are required for packaging of the RNA replicon, and typically include the capsid protein, E1 glycoprotein, and E2 glycoprotein in the mature alphavirus (certain alphaviruses, such as Semliki Forest Virus, contain an additional protein, E3, in the mature coat). The term “alphavirus structural protein(s)” refers to one or a combination of the structural proteins encoded by alphaviruses. These are synthesized (from the viral genome) as a polyprotein and are represented generally in the literature as C-E3-E2-6k-E1. E3 and 6k serve as membrane translocation/transport signals for the two glycoproteins, E2 and E1. Thus, use of the term E1 herein can refer to E1, E3-E1, 6k-E1, or E3-6k-E1, and use of the term E2 herein can refer to E2, E3-E2, 6k-E2, or E3-6k-E2.

Embodiments of the invention include an adenovirus based vaccine that elicits an immune response against Eastern Equine Encephalitis Virus (EEEV). The NIAID, in response to this threat, has classified the EEEV organism as a category B organism. Currently there are no commercially available vaccines for protection from EEEV in the instance of a bioterrorism event.

Eastern equine encephalitis (EEE) is a serious and often fatal disease of humans, equines and other domestic animals. Although outbreaks are sporadic both temporally and spatially, and generally involve small numbers of cases, high mortality rates and serious sequelae make EEE a feared disease. Control measures are extensive in many coastal North American communities, making EEE an economically important disease.

The virion capsid is made of three structural proteins (E1, E2, and E3) that compose the envelope of the virus. Studies indicate that these structural proteins are vital to virus replication and have also been found to be highly antigenic. While variation does exist between the North and South American strain of EEEV, the inventors have found, as well as others, that the North American strain poses the more interesting target strain because the clinical pathology demonstrate that the disease is highly fatal upon exposure to humans. No vaccine is currently available to treat EEEV. Attempts at producing such a vaccine have been problematic. In certain validation studies the inventor have or will use mouse and hamster models of EEE. Mouse and hamster are validated models for EEE and have been shown to mirror both the infectivity and the fatality of human EEE.

Virion Structure: Alphavirus virions are about 70 nm in diameter and are composed of an icosahedral nucleocapsid with T=4 symmetry, surrounded by a lipid envelope containing glycoprotein spikes. The three-dimensional organization of viral particles has been determined for Ross River virus (RRV) (Cheng et al., 1995), Sindbis virus (Paredes et al., 1993) and VEE (Paredes et al., 2001) that represent three different alphavirus complexes. Electron cryomicroscopy and image reconstruction demonstrated that for these viruses the envelope glycoproteins are arranged on the outer surface of particles in a very similar way. The 240 heterodimers of E1 and E2 are combined into 80 trimers forming glycoprotein spikes distributed on the viral particle surface in a T=4 icosahedral lattice that mirrors the nucleocapsid symmetry. The E1 glycoprotein appears to lie parallel to the lipid envelope (Lescar et al., 2001) while the E2 glycoprotein projects outward from the virion to form the spikes (Pletnev et al. 2001).

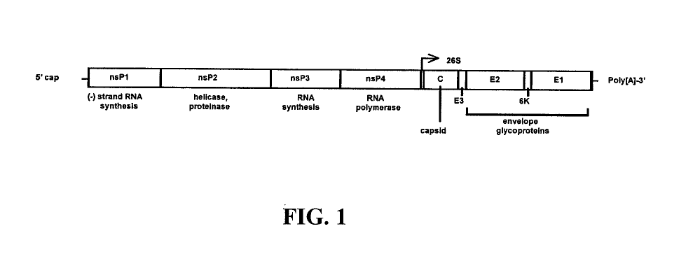

Genetics and Replication: Alphaviruses have plus or messenger sense RNA genomes encoding four nonstructural proteins (nsP 1-4) and three structural proteins (Capsid, E1 and E2 glycoproteins) (FIG. 1). Alphaviruses enter the cytoplasm via receptor-mediated endocytosis (Griffin, 2001). The E2 protein is believed to interact with cellular receptors, and the E1 protein mediates fusion with endosomal membranes.

Virion Assembly and Antigenic Properties: Formation of alphavirus particles is based on specific RNA-protein and protein-protein interactions. During the final steps of viral replication, the capsid protein molecules in completely or partially assembled nucleocapsids interact with the cytoplasmic domain of E2 in E1-E2 heterodimers on the cell membrane. Because of their close association, E1 and E2 share some biological functions and antigenic properties. E2 elicits high-titer, virus-specific, neutralizing antibodies. Two domains important for neutralizing antibody have been identified in the E2 glycoprotein: amino acids 114-120 (mutations in this peptide cause strong conformational changes in E2) and, 180-220 (or 230-250 for Ross River (RR) virus) a linear epitope that binds neutralizing monoclonal antibodies (Johnson et al., 1990). Antibodies to the amino terminal-proximal domain neutralize virus infectivity by blocking virion attachment to susceptible cells (Roehrig et al., 1988). Antibodies to the E2 glycoprotein can also block virus hemagglutination, presumably because of its close association with E1. The amino terminal 25 amino acids of the VEE virus E2 glycoprotein protect mice from virus challenge (Hunt and Roehrig, 1995). Mutations in the E2 glycoprotein have been associated with differences in virulence (propulic et al., 1997; Kinney et al., 1993; Tucker and Griffin, 1991) and the ability to replicate in mosquitoes (Brault et al., 2002; Woodward et al., 1991).

North and South American Strains: North and South American strains were first distinguished using kinetic hemagglutination-inhibition (HI) studies, and later monoclonal antibodies (MAbs). One MAbs that specifically recognizes an E1 glycoprotein epitope of North American isolates, regardless of year or location of isolation, demonstrated antigenic conservation within North America. In contrast, South American EEEV exhibit greater antigenic diversity as demonstrated by the inability to produce a South American-specific Mab. Humans receiving the formalin-inactivated EEEV vaccine, made from a North American strain, develop neutralizing antibodies against North but not South American strains, further supporting their antigenic distinction.

Clinical Disease and Pathogenesis: EEEV causes high rates of mortality in humans, equines, chickens (Guy et al., 1994), turkeys, emus, whooping cranes and pigs (Weaver, 2001). In North America, most human infection is subclinical or unapparent. Early symptoms typically include fever, headache, myalgias, photophobia and dysthesias. Fifty to 90% of apparent cases proceed to encephalitis characterized by irritability, restlessness, headache, drowsiness, anorexia, diarrhea, convulsions, and coma. The fatality rate is higher in patients over 10 years of age, but surviving children generally suffer more severe sequelae. Death due to encephalitis usually occurs 2-10 days after the onset of signs and symptoms, and survivors generally suffer progressive, disabling mental and physical sequelae. In 36 human cases reported in the United States between 1988 and 1994, the mortality rate was 36%, and 35% of survivors were moderately or severely disabled. Patients who suffer permanent neurological sequelae usually live a normal life span, but are incapable of sustaining gainful employment.

Pathogenesis of EEEV infections leading to invasion of the human CNS is poorly understood. Clinical studies using magnetic resonance imaging and computed tomography show changes in the basal ganglia and thalami, suggesting brain edema, ischemia and hypoperfusion in the early stage of disease (Deresiewicz et al., 1997). Gross pathological investigations from fatal cases report brain edema with necrosis, facial or generalized edema, vascular congestion and hemorrhage in the brain and visceral organs (Azimi, 1997; Bastian et al., 1975; Farber et al., 1940; Femster, 1957; Femster, 1938; Getting, 1941; Jordan and McCrumb, 1965).

Mice (Liu et al., 1970), hamsters (Paessler et al., 2004), guinea pigs (Walder et al., 1980) and rhesus monkeys (Nathanson et al., 1969) have been used for experimental EEE studies and histopathological studies of equine (Del Piero et al., 2001) and porcine (Elvinger et al., 1994) cases are available. The mouse model for alphavirus encephalitis is well established for several members of the genus (Charles et al., 1995, although it generally lacks the ability to reproduce the vascular component of EEE (Liu et al., 1970). Murine models for EEE (Liu et al., 1970) reproduce the lymphoid involvement and cerebral pathology, but hamsters more faithfully reproduce the vasculitis associated with micro-hemorrhages in the brain, which dominates the pathological picture in fatal human EEE (Paessler et al., 2004). Most strains of EEEV produce high rates of fatal encephalitis in peripherally infected mice (Liu et al., 1970; Schoepp et al., 2002). Initial murine replication is detected in fibroblasts, osteoblasts and skeletal muscle myocytes near the site of subcutaneous (sc) infection (Vogel et al., 2005). Virus is first detected in the brain one day after sc infection, with rapid interneuronal spread leading to direct neuronal death by day 4. Invasion of the CNS by EEEV probably occurs by a vascular route, rather than via peripheral nerves or the olfactory bulb like VEEV. In hamsters, neuronal cell death is detectable only in late stages of disease after EEEV replicates in a variety of visceral organs, produces viremia, and penetrates the brain (Paessler et al., 2004). The pathological manifestations and antigen distribution in the brain are similar to those described in human EEE.

EEEV Vaccines: A formalin-inactivated vaccine was developed several decades ago at USAMRIID from a wild-type, virulent strain of EEEV (Bartelloni et al., 1970; Maire et al., 1970) and has been administered to laboratory personnel at risk under an IND protocol. This vaccine requires three doses over a 35 day period for generation of protective immunity. In addition, immunity is short-lived and frequent boosters are required to maintain a protective level of neutralizing antibodies (a 1:20 plaque reduction neutralizing (PRNT) antibody titer is recommended by USAMRIID). Antibodies generated by this vaccine react strongly in Western blot assays with both of the envelope (E1, E2) glycoproteins of North American strains, but reactivities with the glycoproteins of South American strains are substantially weaker, with a modest to virtual lack of reactivity with the E2 protein of the latter (Strizki and Repik, 1995). Comparable EEEV veterinary vaccines have been produced and administered to a variety of domestic animals (Jochim and Barber, 1974; Snoeyenbos et al., 1978), including in multivalent preparations with VEEV and WEEV (Barber et al., 1978). These vaccines fail to protect horses in the Florida strain unless administered twice per year. A recent equine case of EEE was undoubtedly caused by an incompletely inactivated equine vaccine lot (Franklin et al., 2002), underscoring the risk of inactivated alphavirus vaccines made from virulent strains. Multivalent vaccines derived from extracted envelope glycoproteins of these alphaviruses are also immunogenic in mice (Pedersen, 1976). Development of the next generation of EEEV vaccines that can be administered quickly and can safely elicit a protective response in a broad range of recipients is a high priority.

Protective Alphavirus Determinants: Mapping of EEEV epitopes has been conducted using monoclonal antibodies and competition binding studies. The E2 protein contains at least 7 partially overlapping antigenic sites. MAbs to sites E2-2 and E2-3 neutralize viral infectivity. Only MAbs to sites E2-2, -3, and -7 protect mice against lethal infection (Pereboev et al., 1993). The closely related VEEV has been more thoroughly studied using peptides spanning the entire E2 protein. The principal neutralization site is composed of several conformationally stable, discontinuous epitopes mapped to the E2 protein (Pereboev et al., 1996; Roehrig and Mathews, 1985), and passively transferred MAbs that react with these epitopes protect mice against lethal challenge (Mathews and Roehrig, 1982). Epitopes cross-reactive with EEEV are found on the E2 and E1 proteins, but none is neutralizing (Pereboev et al., 1996). Vaccination with a peptide composed of the amino-terminal 25 amino acids of the VEEV E2 protein protects mice from challenge (Hunt et al., 1990), and viral replication in peptide-immunized animals is limited in comparison to lethal infection of nonimmunized controls (Hunt et al., 1991). Although polyclonal antipeptide sera and a monoclonal antipeptide antibody are normeutralizing, they passively protect naive mice from challenge. Another peptide (amino acids 241-265) protects 60-70% of VEEV-challenged mice (Johnson et al., 1991).

Neutralization escape variants selected by anti-E2 MAbs block viral hemagglutination, and passively protect mice. Mutations in escape variants cluster in E2 amino acids 182-207 (Johnson et al., 1990).

C. Emerging or Re-Emerging Diseases

While illnesses like influenza can be prevented to a certain extent by immunization. Diseases like bird flu and SARS and other emerging infectious diseases, which have no available vaccine and are associated with a substantial mortality rate, present grave risks. There is increasing concern about the possibility of the sudden emergence of a highly transmissible and highly pathogenic influenza virus, or other microbes. In addition to well-recognized endemic and epidemic viruses, emerging viral infections have been important causes of pneumonia. For example, a hantavirus pneumonia syndrome was recognized in the American Southwest in 1993 with a case-fatality rate of 37%. In 2003, the SARS virus apparently jumped from bats to civets to humans in China, causing more than 8,000 cases of pneumonia worldwide with a case-fatality rate of 10%. Based on these occurrences, it is reasonable to expect that additional emergent microbial infections will be identified in the future. In addition, both hantavirus and SARS virus are classified as Category C bioweapon agents.

Avian Flu (H5N1) The threat of a human influenza pandemic has been increasingly publicized over the past several years with the emergence of highly virulent avian influenza viruses, notably H5N1 viruses, which have infected humans in several Asian and European countries. Embodiments of the invention contemplate the production of an adenoviral based vaccine for the treatment and/or prevention of infection by various forms of Avian Flu, such as H5N1.

Present day flu vaccines typically contain hemagglutinin and neuraminidase proteins of Influenza virus. Influenza A virus is further subdivided into subtypes based on the antigenic composition (sequence) of hemagglutinin (H1-H15) and neuraminidase (N1-N9) molecules. Representatives of each of these subtypes have been isolated from aquatic birds, which probably are the primordial reservoir of all influenza viruses for avian and mammalian species. Transmission has been shown between pigs and humans and, recently (H5N1), between birds and humans.

Three types of inactivated influenza vaccine are currently used in the world: whole virus, split product, and surface antigen or “subunit” vaccines. These vaccines all contain the surface glycoproteins, hemagglutinin (HA) and neuraminidase (NA) of the influenza virus strains.

It is contemplated that all or part of a HA and/or NA may be used in the context of the present invention to provide an adenoviral based vaccine for the treatment and prevention of Avian Flu or other similar types of influenza virus.

Severe acute respiratory syndrome (SARS) SARS captured the attention of the world in the Spring of 2003. The syndrome arose in China in late 2002 and was spread around the world by travelers. Nearly 800 people were killed and 8,000 infected during the initial outbreak. This previously unknown disease has had its most severe impact in Hong Kong and China, but has also been identified in patients in Canada, the United States, Europe and other Asian countries. The rapid global spread of the disease highlights the increasing risk for pandemics created by increasing globalization and travel. An individual infected with a disease can easily travel to any major city in a matter of hours.

One target for neutralizing SARS is the receptor binding protein S, or Spike (Bisht et al., 2004; Yang et al., 2004b; ter Meulen et al., 2004). The S protein is a 150 to 180 kDa highly glycosylated trimeric class-I fusion protein (Bosch et al., 2003; Song et al., 2004) responsible for receptor binding and virus-membrane fusion and tissue tropism of coronaviruses. Immunization with gene or viral vectors encoding fragments or full-length S-proteins induce SARS-CoV nAb (Sui et al., 2004; Zeng et al., 2004; Zhang et al., 2004) and protection (Buchholz et al., 2004; Bukreyev et al., 2004; Yang et al., 2004b). Both the putative S1 (Sui et al., 2004; Zeng et al., 2004) and S2 subunits (Zeng et al., 2004; Zhang et al., 2004) of S are immunogenic. Several vaccine approaches have been described for SARS, including whole inactivated virus (WIV) (Takasuka et al., 2004), DNA (Yang et al., 2004b; Zeng et al., 2004) and viral vectors (Bisht et al., 2004; Bukreyev et al., 2004; Gao et al., 2003). Although such vaccines induce a specific, neutralizing immune response there are safety concerns with respect to use in humans. Embodiments of the invention contemplate using an adenoviral based vaccine delivery platform to produce a relatively safe vaccine for the treatment and prevention of SARS infection, as well as infection by other cornavirus (see U.S. Patent Application publication number 2006093616, which is incorporated herein by reference).

D. Toxins

There are numerous toxin to which vaccines and/or antitoxins can be produced, including, but not limited to, tetanus toxin, diphtheria toxin, pertussis toxin (PT), cholera toxin (CT), the E. coli heat-labile toxins (LT1 and LT2), Pseudomonas endotoxin A, Clostridium botulinum C2 and C3 toxins, as well as toxins from C. perfringens, C. spiriforma, C. difficile, and Bacillus anthracis.

### II. Adenoviral Vaccines

Vaccination has not only been one of the most significant advancements in healthcare, but also a cost-effective means of public health intervention. While conventional vaccine strategies have focused on live-attenuated or killed virus approaches, a new approach to the development of vaccines utilizes platform technologies and scientific advancement to overcome the challenges in vaccine design. Methods and compositions of the present invention include the construction and verification of adenoviral vaccines that elicit an immune response against biological warfare agents such as, but not limited to Eastern Equine Encephalitis Virus (EEEV) and Yersinia pestis, as well as other agents described herein. The inventors combine commercial scale process development and cGMP manufacturing infrastructure with access and expertise related to BSL-4 facilities, to generate a multi-use vaccine platform applicable to biodefense, emerging, and re-emerging infectious diseases.

There has been considerable effort expended in studying and improving the stability of its adenoviral based products. The existing stability data for products in late phase clinical trials confirms that an exemplary adenovirus vector used in this project is stable in DPBS pH 7.4, 10% glycerin formulation buffer at ≦−60° C., with over 6 years of data showing no adverse trends in stability test results. More recent stability studies examining multiple formulations at 1×10¹¹ viral particles/mL show stable product up to 18 months at 4° C. (data not shown). The stability study results are important to the development of a viable vaccine product that can be stored at room temperature. These studies are important because it is anticipated that one will be using a lower virus titer formulation, for example 1×10¹¹ viral particles/mL, and the adenovirus vaccine will potentially be stored at room temperature to meet stockpile needs.

Compositions of the invention may be stored at least about, at most about, or about 0, 1, 2, 3, 4, 5, 10, 20, 30° C. more or less for at least 1, 5, 10, 15, 20, 25, 30 months or more. In other aspects of the invention compositions may be stored at room temperature for at least 7 days, 14 days, 21 days, 1, 5, 10, 15, 20, 25, 30 months or more. Compositions to the invention can be manufactured at commercial scale and may remain viable and stable for time periods that allow stockpiling of the compositions.

A. Adenoviral Vaccines

Aspects of the invention include the development, the production, and the evaluation of a vaccine comprising one or more antigenic determinants of one or more pathogenic organisms. In particular aspects antigenic determinants include EEEV and Y. pestis polypeptide segments encoded by an adenoviral vector system. The adenoviral vector system not only provides a viable delivery vehicle, but also may elicit an immune response. Aspects of the invention use a replication-defective adenovirus type-5 vector, which has been used in a wide range of doses, with minimal toxicity. Alternatively, replication competent adenoviral vectors may also be used, including conditionally replication competent adenoviral vectors. The adenovirus is well-established for use in gene transfer in several therapeutic applications including anti-cancer immunotherapy and cardiovascular revascularization. While a common argument against adenoviral vectors is the potential for pre-existing immunity, the vector has been shown to express high levels of its target gene without any limitations due to neutralizing antibodies, which allows a vector to be used in the same person for multiple indications. However, alternative adenoviral serotypes may be used. The functionality of the vector is significant for first responders and emergency situations where a first line of defense can be achieved through immediate vaccination. Further, some of the vectors have been shown to be replication-defective, unable to integrate into the host chromosome, and capable of inducing a robust and long-lived expression of a gene in animal models.

Aspects of the invention include: 1) the construction and scale up of vaccine vectors for Good Manufacturing Procedure (GMP) production. (2) the selection and insertion of one or more antigens from one or more organisms into a replication-defective or replication competent adenovirus backbone to generate the adenovirus-vaccine vector. The resulting product will be purified, characterized, and sequenced. Certain aspects of the invention will include characterization of the product; feasibility studies, and GLP-grade material for vaccination studies.

Compositions of the invention can be assessed using mouse animal model efficacy/toxicology studies. Animal models are typically used in evaluating the safety, immunogenicity, and protection of a vaccine. A small animal model is needed for initial studies, due to the large number of animals required to show statistical relevance. Also, the mouse model has been shown in previous studies as an efficient means for testing EEEV protection that is similar to the protection anticipated in humans.

Compositions of the invention will be adapted for GMP production of vaccines. Regulatory requirements will be satisfied and current Good Manufacturing Practices (cGMP) adapted for production and human testing of inventive compositions. These procedures typically include document preparation and review; GMP production of the product as well as a master cell and virus banks; filling of the product; and release testing.

Evaluation of vaccines can be performed in golden Syrian hamster animal models. Testing for efficacy in humans is typically not possible. Accordingly, the inventors will, in some instances use the golden Syrian hamster model as an alternative or additional animal model for high-priority agents. Based on previous studies the Syrian hamster model has been shown to be an effective means for testing EEEV due to its 100% mortality upon exposure and histopathological disease that closely resembles human EEE.

The adenovirus is an attractive delivery system. Embodiments of the invention can utilize a suspension cell process with average yields of 1×10¹⁶ viral particles per batch. The process can be free of or essentially free of protein, serum, and animal derived components making it suitable for a broad range of both prophylactic and therapeutic vaccine products. Current manufacturing process can be utilized to supply Phase I-III clinical materials and is scaleable to commercial production volumes.

Aspects of the invention include compositions that are stable and easy to transport. The stability of the adenovirus products will allow for stockpiling as well as easy distribution in emergency situations. Further aspects include storage of the inventive compositions at room temperature. The compositions of the invention can also include lyophilized compositions. Furthermore, adenoviral vaccines allow for multiple delivery methods such as intramuscular, nasal, mucosal, and subcutaneous delivery.

The genomes of alphaviruses, including EEEV, can be placed into infectious cDNA clones to allow virtually any genetic manipulation, including the introduction of foreign genes such as toxins or human cytokines. EEEV therefore has the potential to be developed into even more virulent pathogen. Compositions and methods of this invention will provide a valuable first line of defense by deterrence for potential terrorists in search of weapons where no vaccine or treatment option exists. The adaptability of the adenoviral delivery system will also provide dividends in future vaccine development.

In certain embodiments, an adenovirus vector can handle a 6 kb or greater genetic insert and can provide for delivery of multiple antigens which may allow for instances of overcoming resistance or genetically modified organisms or weapons. For instance, a multivalent vaccine may be produced to provide cross protection, for example, protection against Venezuelan, Eastern, and Western Equine Encephalitis Viruses. The multivalent product would allow for a singe vaccine regime for a variety of bioterrorism threats. Further, insertion of a nucleic acid encoding one or more antigens providing an immune response to one or more organisms facilitates construction of vaccine(s) against multiple targets. Progress in this area of development may also pave the way for quick responses to emerging infectious diseases (i.e., SARS or Avian Flu) with no pre-existing treatment options.

A number of adenoviral vectors have been constructed using homologous recombination. A number of these adenoviral vectors have been used in clinical studies. Typically shuttle vectors are used for generation of adenovirus vector, it is contemplated that construction and subsequent expression characteristics of the adenoviral vaccines may use those techniques described herein in combination with those know in the art to produce the adenoviral vectors of the invention.

Still further embodiments of the invention will include stable adenoviral-based products. In particular aspects, an adenoviral vaccine of the invention will include a heterologous nucleic acid segment in a deleted early region of an adenovirus, for example the adenoviral E1, E2A, E2B, E3, E4 region or combinations thereof. Existing stability data for adenoviral products currently in late phase clinical trials are indicative of the stability of the adenoviral vaccines described in this application. For example, current adenoviral preparations in DPBS at pH 7.4 in a 10% glycerin formulation buffer at ≦−60° C., with over 6 years of data showing no adverse trends in stability. Recent stability studies examining multiple formulations at 1×10¹¹ viral particles/mL show stable product up to 18 months at 4° C. The stability studies are important to the development of a viable vaccine product that can be stored at room temperature.

B. Expression Cassettes

In certain embodiments of the present invention, the methods set forth herein involve nucleic acid sequences wherein the nucleic acid is comprised in an “expression cassette.” Throughout this application, the term “expression cassette” is meant to include any type of genetic construct containing a nucleic acid coding for a gene product (e.g., an antigenic determinant) in which part or all of the nucleic acid encoding sequence is capable of being transcribed.

Promoters and Enhancers—In order for the expression cassette to effect expression of a transcript, the nucleic acid encoding gene will be under the transcriptional control of a promoter. A “promoter” is a control sequence that is a region of a nucleic acid sequence at which initiation and rate of transcription are controlled. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a cell in a subject is contemplated as a promoter that can be applied in the methods and compositions of the present invention. One of ordinary skill in the art would be familiar with the numerous types of promoters that can be applied in the present methods and compositions. In certain embodiments, for example, the promoter is a constitutive promoter, an inducible promoter, or a repressible promoter. The promoter can also be a tissue selective promoter. A tissue selective promoter is defined herein to refer to any promoter which is relatively more active in certain tissue types compared to other tissue types. Examples of promoters includes the CMV promoter.

The promoter will be one which is active in a cell and expression from the promoter results in the presentation of an antigenic determinant to a subject's immune system. For instance, where the cell is an epithelial cell the promoter used in the embodiment will be one which has activity in that particular cell type.

A promoter may be one naturally associated with a gene or sequence, as may be obtained by isolating the 5′-non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™ (see U.S. Pat. Nos. 4,683,202 and 5,928,906, each incorporated herein by reference).

Naturally, it will be important to employ a promoter and/or enhancer that effectively directs the expression of the DNA segment in the cell type, organelle, and organism chosen for expression. Those of skill in the art of molecular biology generally understand the use of promoters, enhancers, and cell type combinations for protein expression, for example, see Sambrook et al. (2001), incorporated herein by reference. The promoter may be heterologous or endogenous.

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used. The use of other viral or mammalian cellular or bacterial phage promoters, which are well-known in the art to achieve expression of polynucleotides, is contemplated as well, provided that the levels of expression are sufficient to produce an immune response.

Additional examples of promoters/elements that may be employed, in the context of the present invention include the following, which is not intended to be exhaustive of all the possible promoter and enhancer elements, but, merely, to be exemplary thereof.

Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990); Immunoglobulin Light Chain (Queen et al., 1983; Picard et al., 1984); T Cell Receptor (Luria et al., 1987; Winoto et al., 1989; Redondo et al.; 1990); HLA DQ a and/or DQ β (Sullivan et al., 1987); β Interferon (Goodbourn et al., 1986; Fujita et al., 1987; Goodbourn et al., 1988); Interleukin-2 (Greene et al., 1989); Interleukin-2 Receptor (Greene et al., 1989; Lin et al., 1990); MHC Class II (Koch et al., 1989); MHC Class II HLA-DRa (Sherman et al., 1989); β-Actin (Kawamoto et al., 1988; Ng et al.; 1989); Muscle Creatine Kinase (MCK) (Jaynes et al., 1988; Horlick et al., 1989; Johnson et al., 1989); Prealbumin (Transthyretin) (Costa et al., 1988); Elastase I (Omitz et al., 1987); Metallothionein (MTII) (Karin et al., 1987; Culotta et al., 1989); Collagenase (Pinkert et al., 1987; Angel et al., 1987); Albumin (Pinkert et al., 1987; Tronche et al., 1989, 1990); α-Fetoprotein (Godbout et al., 1988; Campere et al., 1989); t-Globin (Bodine et al., 1987; Perez-Stable et al., 1990); β-Globin (Trudel et al., 1987); c-fos (Cohen et al., 1987); c-HA-ras (Triesman, 1986; Deschamps et al., 1985); Insulin (Edlund et al., 1985); Neural Cell Adhesion Molecule (NCAM) (Hirsh et al., 1990); α1-Antitrypsin (Latimer et al., 1990); H2B (TH2B) Histone (Hwang et al., 1990); Mouse and/or Type I Collagen (Ripe et al., 1989); Glucose-Regulated Proteins (GRP94 and GRP78) (Chang et al., 1989); Rat Growth Hormone (Larsen et al., 1986); Human Serum Amyloid A (SAA) (Edbrooke et al., 1989); Troponin I (TN I) (Yutzey et al., 1989); Platelet-Derived Growth Factor (PDGF) (Pech et al., 1989); Duchenne Muscular Dystrophy (Klamut et al., 1990); SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988); Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell and/or Villarreal, 1988); Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989); Papilloma Virus (Campo et al., 1983; Lusky et al., 1983; Wilkie, 1983; Spalholz et al., 1985; Lusky et al., 1986; Cripe et al., 1987; Gloss et al., 1987; Hirochika et al., 1987; Stephens et al., 1987); Hepatitis B Virus (Bulla et al., 1986; Jameel et al., 1986; Shaul et al., 1987; Spandau et al., 1988; Vannice et al., 1988); Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989); Cytomegalovirus (CMV) (Weber et al., 1984; Boshart et al., 1985; Foecking et al., 1986); Gibbon Ape Leukemia Virus (Holbrook et al., 1987; Quinn et al., 1989).

Enhancers were originally detected as genetic elements that increased transcription from a promoter located at a distant position on the same molecule of DNA. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have very similar modular organization. Additionally, any promoter/enhancer combination (as per the Eukaryotic Promoter Data Base EPDB) could also be used to drive expression of a gene. Further selection of a promoter that is regulated in response to specific physiologic signals can permit inducible expression of a construct. For example, with the polynucleotide under the control of the human PAI-1 promoter, expression is inducible by tumor necrosis factor. Examples of inducible elements, which are regions of a nucleic acid sequence that can be activated in response to a specific stimulus include (Element/Inducer): MT II/Phorbol Ester (TFA) or Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)/Glucocorticoids (Huang et al., 1981; Lee et al., 1981; Majors et al., 1983; Chandler et al., 1983; Ponta et al., 1985; Sakai et al., 1988); β-Interferon/poly(rI)x or poly(rc) (Tavernier et al., 1983); Adenovirus 5 E2/E1A (Imperiale et al., 1984); Collagenase/Phorbol Ester (TPA) (Angel et al., 1987a); Stromelysin/Phorbol Ester (TPA) (Angel et al., 1987b); SV40/Phorbol Ester (TPA) (Angel et al., 1987b); Murine MX Gene/Interferon, Newcastle Disease Virus (Hug et al., 1988); GRP78 Gene/A23187 (Resendez et al., 1988); α-2-Macroglobulin/IL-6 (Kunz et al., 1989); Vimentin/Serum (Rittling et al., 1989); MHC Class I Gene H-2κb/Interferon (Blanar et al., 1989); HSP70/E1A, SV40 Large T Antigen (Taylor et al., 1989, 1990a, 1990b); Proliferin/Phorbol Ester-TPA (Mordacq et al., 1989); Tumor Necrosis Factor/PMA (Hensel et al., 1989); and Thyroid Stimulating Hormone a Gene/Thyroid Hormone (Chatterjee et al., 1989).

Initiation Signals—A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

IRES—In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages (see U.S. Pat. Nos. 5,925,565 and 5,935,819).

Multiple Cloning Sites—Expression cassettes can include a multiple cloning site (MCS), which is a nucleic acid region that contains multiple restriction enzyme sites, any of which can be used in conjunction with standard recombinant technology to digest the vector.

Polyadenylation Signals—In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

Other Expression Cassette Components—In certain embodiments of the invention, cells infected by the adenoviral vector may be identified in vitro by including a reporter gene in the expression vector. Generally, a selectable reporter is one that confers a property that allows for selection. A positive selectable reporter is one in which the presence of the reporter gene allows for its selection, while a negative selectable reporter is one in which its presence prevents its selection. An example of a positive selectable marker is a drug resistance marker (genes that confer resistance to neomycin, puromycin, hygromycin, DHFR, GPT, zeocin and histidinol). Other types of reporters include screenable reporters such as GFP.

Embodiments of the invention can use current adenoviral platform technologies designed to create vaccines by preparing an adenoviral nucleic acid comprising a heterologous nucleic acid segment that encodes an antigen related to a pathogen. Aspects of the adenoviral vaccine construction include inserting genetic material into an adenoviral vector and confirming the construct through characterization and sequencing of the nucleic acid, virus and virus product. The adenoviral vaccine is then put through a series of feasibilities studies designed to assess scalability.

Construction. A nucleic acid segment is inserted into a replication-defective adenovirus backbone via insertion into a shuttle vector followed by a standard recombination protocol to generate the adenovirus vaccine vector. Briefly, 293INT cells, or another suitable cell line, are cultured for a suitable time period after which the cells are infected with a shuttle vector. Infected cells are further cultured and monitored for the development of plaques. The infection and plaque formation cycle is estimated to take 7 to 10 days, and a single round of plaque purification can be used.

In certain aspects, the design of an adenoviral-EEEV vaccine may rely on two proven strategies for alphavirus envelope glycoproteins: expression of an entire E3-E2-6K-E1 encoding nucleic acid, as demonstrated previously using an adenovirus vector to protect mice against VEEV (Phillpotts et al., 2005). This region of the VEEV genome is also highly immunogenic and protective when expressed by a baculovirus (Hodgson et al., 1999); and expression of E3-E2 alone, which, when expressed by baculoviruses, is incompletely processed but provides complete protection in the VEEV system (Hodgson et al., 1999).

C. Bacterial Vaccines

Further embodiments of the invention include the construction of an adenoviral vaccine candidate that elicits a potent immune response against pathogenic bacteria, such as Yersinia pestis, the causative agent of plague. The plague vaccine was selected as an exemplary use of adenoviral based vaccines because of the growing concern surrounding the its use in a potential terrorism event. The NIAID, in response to this threat, has classified the organism as a Category A priority organism. Currently there are no commercially available vaccines for protection from plague in the instance of a bioterrorism event. The lack of a vaccine is particularly worrisome because of the pathogen's capability to inflict widespread morbidity and mortality, upon exposure, to both civilians and military personnel. Aspects of the invention include the development, production, and evaluation of a vaccine featuring the low calcium response V antigen (LcrV) alone or in combination with two other protective Y. pestis antigens Caf1 (capsular antigen) and YscF (a type 3 secretion system structural protein) in an adenoviral vector system. The adenoviral vector system was selected because it not only provides a viable delivery vehicle, but also because of the ability of the vector to elicit an immune response. Methods and compositions of the invention include, but are not limited to 1) construction of a vaccine and feasibility studies for GMP production, 2) GLP production of the vaccines for animal studies, 3) small animal efficacy studies (mice) and 4) second, confirmatory animal model study (guinea pigs). Both of the animal models are currently being used for inhalational anthrax and plague vaccine studies.

Certain embodiments of the invention include compositions and methods of use for an adenoviral vaccine that elicits an immune response against Yersinia pestis (plague) or other pathogenic bacteria. In certain aspects the invention includes a replication-defective adenovirus type-5 vector, which has been developed for performing gene transfer in several therapeutic applications including anti-cancer immunotherapy and cardiovascular revascularization. While a common argument against adenoviral vectors is the potential for pre-existing immunity, it has shown that exemplary adenoviral vectors express high levels of target gene without any limitations due to neutralizing antibodies. This same vector and other analogous vectors can be used in the same person for multiple indications. The functionality of the vector is significant for first responders and emergency situations that can achieve a first line of defense through immediate vaccination. Further, through animal studies, Introgen has shown in non-limiting examples of a vector used in these studies to be replication-defective, unable to integrate into the host chromosome, and capable of inducing a robust and long-lived expression of a gene.

Animal models will be instrumental in evaluating the safety, immunogenicity, and protection of the vaccine candidate. A small animal model is crucial for initial studies, due to the large number of animals required to show statistical relevance. Also, the mouse model has been shown in previous studies to be an efficient means for testing protection against plague. Both bubonic (subcutaneous challenge) and pneumonic (intranasal and aerosol challenges) plague models will be employed.

Due to the unique nature of this product, testing for efficacy in humans is not possible. Accordingly, the project will utilize a guinea pig model as the second part of the two-animal model as dictated for high priority agents. Guinea pigs are exceptionally susceptible to plague and are typically used as a standard animal model to estimate the virulence of Y. pestis strains and to evaluate the protective effect of anti-plague preparations. Both bubonic (subcutaneous challenge) and pneumonic (intranasal and aerosol challenges) plague models will be employed.

D. Nucleic Acids

The present invention concerns nucleic acids that are capable of expressing an antigenic determinant from a pathogenic organism. A DNA segment encoding an antigenic determinant polynucleotide and/or polypeptide refers to a DNA segment that contains wild-type, polymorphic, or mutant an antigenic determinant and/or polypeptide-coding sequences that encode an antigenic determinant. Included within the term “DNA segment” are polynucleotides, DNA segments smaller than a polynucleotide, and recombinant vectors. Recombinant vectors may include plasmids, cosmids, phage, viruses, and the like. In certain embodiments recombinant adenoviruses are contemplated. In particular, an adenovirus comprising an expression cassette or polynucleotide encoding an antigenic determinant is contemplated.

Similarly, a polynucleotide comprising an isolated nucleic acid encoding an atnigenic determinant refers to a DNA segment including all or part of nucleic acid encoding an antigenic polypeptide coding sequences and, in certain aspects, regulatory sequences, isolated substantially away from other naturally occurring genes or protein encoding sequences. The nucleic acid encoding an antigenic determinant may contain a contiguous polynucleotide sequence encoding all or a portion of an antigenic determinant of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, or more nucleotides or base pairs.

The DNA segments used in the present invention may encode biologically functional equivalent antigenic determinants. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to decrease the antigenicity of the protein or to inhibit binding to a given protein.

Additional embodiments of the invention encompass the use of a purified protein composition or a nucleic acid encoding such a protein comprising an antigenic determinant and peptides derived from the amino acid sequence of an antigenic determinant as described herein administered to cells or subjects. Specifically contemplated are antigenic determinants comprise at least 10, 20, 30, 40, 50, 60, 70, 80, 90, 100, 110, 120, 130, 140, 150, 160, 170, 180, 190, 200 or more contiguous amino acids of antigenic determinant described herein.

### III. Pharmaceutical Compositions

The present invention also provides a pharmaceutical composition comprising any composition of the present invention, and a pharmaceutically acceptable carrier. The present invention also provides a vaccine composition comprising any composition of the present invention. The vaccine composition may further comprise at least one adjuvant.

The present invention also provides a method of immunizing a subject, comprising administering to a subject a vaccine composition of the present invention.

According to the present invention, an expression construct encoding an antigenic determinant is administered to a subject to induce an immune response for therapeutic or prophylatic purposes, such as for vaccination against a biological weapon. Thus, in certain embodiments, the expression construct is formulated in a composition that is suitable for this purpose. The phrases “pharmaceutically” or “pharmacologically acceptable” refer to compositions that do not produce adverse, allergic, or other untoward reactions when administered to an animal or a human. As used herein, “pharmaceutically acceptable carrier” includes any and all solvents, carriers, dispersion media, coatings, antibacterial and antifungal agents, isotonic and absorption delaying agents, and the like. The use of such media and agents for pharmaceutically active substances is well known in the art. Except insofar as any conventional media or agent is incompatible with the expression constructs of the present invention, its use in therapeutic compositions is contemplated. Supplementary active ingredients also can be incorporated into the compositions. For example, the supplementary active ingredient may be an additional immunogenic agent or an anti-microbial agent.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. If needed, various antibacterial an antifungal agents can be used, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating compounds in the required amount in the appropriate solvent with various of the other ingredients enumerated above, as required, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically or prophylactically effective. For parenteral administration in an aqueous solution, the solution should be suitably buffered if necessary and the liquid diluent first rendered isotonic with sufficient saline or glucose. These particular aqueous solutions are especially suitable for intravascular and intratumoral administration. In this connection, sterile aqueous media, which can be employed will be known to those of skill in the art in light of the present disclosure.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA.

In some embodiments, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

“Treatment” refers to both therapeutic treatment and prophylactic or preventative measures. Those in need of treatment include those already exposed to a pathogenic microorganism or at risk of such exposure, as well as those in which infection is to be prevented.

Administration—The methods of the present invention pertain to ingestion (such as enteral administration), respiratory administration, or injection of an expression construct encoding an antigenic determinant. Any method of administration known to invoke an immune response is contemplated by the present invention, including oral administration. Oral administration can entail the ingestion of coated capsule or pill that traverses the upper digestive tract intact and releases the adenovirus in the lower digestive tract. For example, an injection can be into the muscle, or enteral administration can be by capsule or muccosal patch, or respiratory administration by inhalation. Administration can also include intravascular administration into one or more arteries or veins.

Dosage—An effective amount of the therapeutic or preventive agent is determined based on the intended goal, for example vaccination against a microorganism. Those of skill in the art are well aware of how to apply gene delivery in vivo and ex vivo situations. For viral vectors, one generally will prepare a viral vector stock. Depending on the kind of virus and the titer attainable, one will deliver at least about, at most about, or about 1×10⁴, 1×10⁵, 1×10⁶, 1×10⁷, 1×10⁸, 1×10⁹, 1×10¹⁰, 1×10¹¹ or 1×10¹² infectious particles, or any value or range there between, to a subject.

The quantity to be administered, both according to number of treatments and dose, depends on the subject to be treated, the state of the subject and the protection desired. Precise amounts of the therapeutic composition also depend on the judgment of the practitioner and are peculiar to each individual.

For example, in some embodiments of the present invention, the dose of viral vector ranges from 1×10¹¹ to 1×10¹⁵ viral particles for injection. In other embodiments, the dose of viral particles per injection is 1×10¹² to 1×10¹⁴. In certain particular embodiments, the dose of viral particles per injection is 1×10¹² to 5×10¹².

### IV. Vaccine Administration

In certain embodiments, the compositions and methods of the present invention involve an adenoviral vaccine, or construct capable of expressing one or more antigenic determinants for the induction of an immune response to one or more pathogenic organisms, can be administered alone or in combination with a second or additional therapy or prophylactic treatment. The methods and compositions including combination therapies enhance the therapeutic or protective effect, and/or increase the therapeutic effect of another anti-pathogen therapy. Therapeutic and prophylactic methods and compositions can be provided in a combined amount effective to achieve the desired effect, such as the killing of a microorganism, inhibiting infection by one or more microorganisms, and/or limiting the growth of a microorganism such that no substantial pathological condition results in a subject exposed to such microorganism(s). This process may involve administering a vaccine to a subject and a second therapy. A subject or microorganism can be contacted with one or more compositions or pharmacological formulation(s) including one or more of the agents (i.e., vaccine or anti-microbial agent), or by contacting subject with two or more distinct compositions or formulations, wherein one composition provides 1) a vaccine; 2) an anti-microbial agent, or 3) both a vaccine and an anti-microbial agent.

A vaccine may be administered before, during, or after an anti-microbial treatment. The administrations may be in intervals ranging from concurrently to minutes to days to weeks. In embodiments where the vaccine is provided to a subject separately from an anti-microbial agent, one would generally ensure that a significant period of time did not expire between the time of each delivery, such that the two compounds would still be able to exert an advantageously combined effect on the subject. In such instances, it is contemplated that one may provide a subject with vaccine and the anti-microbial agent within about 12 to 24 to 72 h of each other and, more preferably, within about 6-12 h of each other. In some situations it may be desirable to extend the time period for treatment significantly where several days (2, 3, 4, 5, 6 or 7) to several weeks (1, 2, 3, 4, 5, 6, 7 or 8) lapse between respective administrations. In certain embodiments the vaccine will be a booster or at least the second exposure of a subject to such a vaccine and can comprise the same or different antigenic determinant relative to an initial administration.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no vaccine or anti-microbial treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the subject, such as their prognosis, strength, health, antibody titer, etc.

Various combinations may be employed. For the example below a vaccine is “A” and an anti-microbial is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present invention to a subject will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies may be applied in combination with the vaccine(s).

In specific aspects, it is contemplated that a standard therapy will include anti-microbial therapy and may be employed in combination with the vaccine compositions and methods as described in this application.

Antimicrobial therapies include, but are not limited to penicillin, ampicillin, bacitracin, carbapenems, cephalosporin, methicillin, oxacillin, vancomycin, chloramphenicol, erythromycin, gentamycin, neomycin, and streptomycin administration.

A. Immunotherapy

The methods and compositions of the invention relate to immunotherapy and vaccination of microbial infection including deliberate infection. In using the immunotherapeutic compositions derived from the polypeptides and peptides of one or more pathogenic organisms, other standard treatments also may be employed. However, it is preferred that the immunotherapy be used alone initially as it effectiveness can be readily assessed. Immunotherapies can broadly be classified as adoptive, passive and active, as described in the following sections.

It is contemplated that a wide variety of infections agents may be treated, prevented, or attenuated using compositions and methods described herein.

Passive Immunotherapy—A number of different approaches for passive immunotherapy exist. They may be broadly categorized into the following: injection of antibodies alone; injection of antibodies coupled to antimicrobial agents; and injection of anti-idiotype antibodies.

Preferably, human monoclonal antibodies are employed in passive immunotherapy, as they produce few or no side effects in a subject. It may be favorable to administer more than one monoclonal antibody directed against two different antigens or even antibodies with multiple antigen specificity. Treatment protocols also may include administration of lymphokines or other immune enhancers. The development of human monoclonal antibodies is described in further detail elsewhere in the specification.

Active Immunotherapy—In active immunotherapy, an antigenic peptide, polypeptide or protein composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993).

Adoptive Immunotherapy—In adoptive immunotherapy, the subject's circulating lymphocytes are isolated in vitro, activated by lymphokines such as IL 2 or transduced with genes for tumor necrosis, and readministered (Rosenberg et al., 1988; 1989). To achieve this, one would administer to an animal, or human patient, an immunologically effective amount of activated lymphocytes in combination with an adenoviral vaccine composition as described herein. The activated lymphocytes will most preferably be the subject's own cells that are activated (or “expanded”) in vitro.

### V. Kits

Kits for implementing methods of the invention described herein are specifically contemplated. Any of the compositions described herein may be comprised in such a kit. In a non-limiting example, an adenoviral vaccine(s), in an aqueous or lyophilized form, and/or a pharmaceutically acceptable buffer are provided in a kit. The adenovirus may also be in the form of a pill, a patch, a nebulizer, a nasal spray, or other formulation or device use to deliver a composition by the methods of administration described herein. In some embodiments, there are kits for vaccination against pathogenic organisms, particularly biological weapons. In these embodiments, a kit can comprise, in suitable container(s) and one or more of 1) a adenoviral vaccine; 2) a means for administration, including a syringe, nebulizer or the like; or 3) pharmaceutically acceptable buffer or delivery vehicle.

Reagents for the vaccination of a subject can comprise one or more of the following: adenovirus encoding one or more antigenic determinants, adjuvants and other immune response enhancing agents, or anti-microbials such as anti-viral agents, anti-biotics, etc.

In certain embodiments the adenoviral vaccine composition may be supplied in a “ready to administer format” where it is allocated in a syringe, a capsule, a pill or other administration device such that the vaccine may be administered in or outside a hospital setting by a physician or a subject.

The components of the kits may be packaged either in aqueous media, in pill or capsule form, or in a lyophilized form. The container means of the kits will generally include at least one vial, test tube, plate, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing vaccines, and any other reagent containers in close confinement for commercial sale. Such containers may include injection or blow molded plastic containers into which the desired vials are retained.

When components of the kit are provided in one and/or more liquid solutions, the liquid solution is typically an aqueous solution that is sterile and proteinase free. In some cases proteinaceous compositions may be lyophilized to prevent degradation and/or the kit or components thereof may be stored at a low temperature (i.e., less than about 4° C.). When reagents and/or components are provided as a dry powder and/or tablets, the powder can be reconstituted by the addition of a suitable solvent. It is envisioned that the solvent may also be provided in another container means.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### EEEV

EEE Animal Models: Mouse and hamster models for human EEE were validated using a variety of EEEV strains and both outbred and inbred mice. Rates of encephalitis and fatality have been determined following sc infection. Although various ages of mice have been challenged, only data from adult (5-13 week-old) mice are presented. Using sc doses of 10³ PFU per mouse, mortality rates are generally 75-100%, with 100% of animals developing disease detectable by day 7. Higher doses administered sc or ip result in 100% mortality. The ability to challenge with either 100% fatal doses or those generating partial survival allows an increased power in challenge studies (with high doses) or a more accurate model human infections (with lower doses), which are not 100% fatal. In hamsters (Table 1), mortality is 100% and histopathological disease closely resembles human EEE (Paessler et al., 2004).

TABLE 1
Mortality of laboratory animals infected
with North American strains of EEEV.

| | | | Log10 | | |
|---|---|---|---|---|---|
| | Age | EEEV | Dose | | |
| Animal | (weeks) | strain | (PFU) | Route | Mortality |
| Hamster | 6 | 792138 | 3 | Sc | 100% |
| NIH Swiss | 5-7 | 792138 | 3 | Sc | 80-100% |
| mice | | | | | |
| | 5-7 | 783372 | 3 | Sc | 80-100% |
| | 9 | FL93-939 | 6 | Sc | 100% |
| | | (clone derived) | | | |
| CD-1 mice | 6 | 792138 | 3 | Sc | 100% |
| | 11 | 792138 | 3 | Sc | 75% |
| C57 BL/6 | 6 | 792138 | 3 | Sc | 75% |
| mice | | | | | |
| 129 Sv/Ev | 10-13 | 792138 | 3 | Sc | 80-90% |
| mice | | | | | |
| NIH Swiss | 6-11 | FL93-939 | 6 | Ip | 100% |
| mice | | (clone derived) | | | |

EEEV strains and clones: In preliminary studies, mice were challenged with a variety of EEEV strains including both North and South American subtypes. However, epidemiologic evidence indicates that the South American strains are probably avirulent for humans (Scott and Weaver, 1989; Weaver, 2001). The inventors developed cDNA clones for two EEEV strains (a North American and a South American strain) so that unlimited supplies of genetically defined virus stocks can be produced without cell culture or animal passages that might result in attenuation or alterations in pathogenesis. Antigenic determinants of alphavirus can be derived from the protein(s) encoded by viruses with sequences provided in GenBank accession numbers L01442, L01443, AF375051, U55350, U55360, AF004459, AF004458, AF1005660, L00930, U55362, DQ138312a, DQ138313a, DQ390224a, DQ138314a, U34999, AF075252, AF075251, AF075253, AF075254, U94612, DQ228210a, U01034, X63135, EF034078, EF034076, EF034077, EF034079, U94602, DQ138315a, DQ138316a, DQ138317a, DQ138318a, DQ138319a, DQ138320a, AF079456, M20162, Z48163, AF214040, AF109297, AY348559, U94609, AF126284, U94608, U94606, or J02363, each of which is incorporated herein by reference in its entirety.

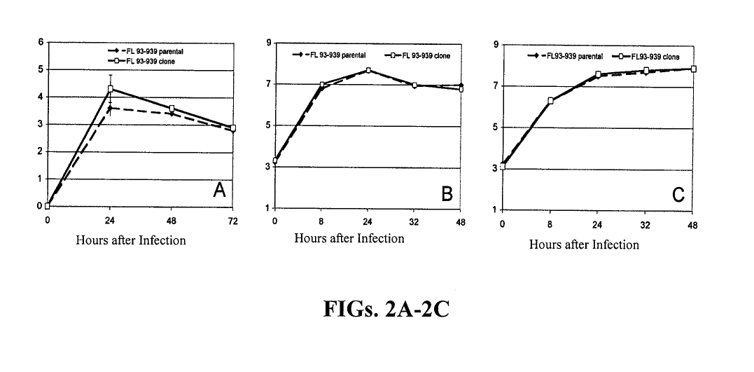

Beginning with RNA extracted from the second passage of a 1993 Florida mosquito isolate, PCR amplicons overlapping unique restriction sites were generated using high fidelity RT-PCR and subcloned. Two or more clones were sequenced and compared to the consensus sequence (PCR amplicons sequenced directly) to ensure that PCR errors were not introduced. The complete genome was then cloned into a low copy vector downstream of a T7 promoter, with a unique Not I restriction site downstream of the poly(A) tract for linearizing plasmid DNA. Capped RNA transcribed from the linearized clone was electroporated into BHK cells and the supernatant was collected 24 hours later when CPE was detected. Plaque forming virus of titer 2×10⁸ PFU/ml was recovered. To ensure the phenotypic fidelity of the virus, it was compared with its parent and other wild-type EEEV strains for Vero, BHK and C7/10 mosquito cell replication, and was found to be indistinguishable (data not shown); murine viremia and virulence were also similar (FIG. 2).

Purification, Characterization, and Sequencing. Typically, after formation of adenovirus plaques, 5 to 10 plaques are picked, DNA purified from the progeny virus, and the DNA analyzed by one or more nucleic acid analysis techniques, such as PCR. Some number of these plaques will be identified as non-recombinant. Of this group of non-recombinants, up to three plaques will be then further amplified in 293INT cells and again analyzed by the PCR assay to confirm that no recombinants are present. One of these cultures will then be chosen as a master stock, to be used for further work and to generate a Virus Bank. The master stocks will be characterized by various methods typically used to characterize adenoviral stocks. Characterization includes, but is not limited to, the detection of mycoplasma, sterility, particle counts, and infectivity. Characterization is typically performed on 1) the initial construct, 2) the cell bank (CB), 3) the virus bank (VB), and 4) the GLP-grade materials.

Typically one or more nucleic acid assays are developed for the detection of the correct sequence for the adenoviral vaccine. Typically all or part of an adenoviral construct will be sequenced. Alternatively or in combination, heterologous DNA, such as expression cassettes may be sequenced in its entirety.

Scale up of the adenoviral vectors of the invention to GMP production include, but is not limited to one or more of a) multiplicity of infection (MOI) study, b) cell density at time of infection, c) infection temperature, d) confirmation of activity with cell line, e) confirmatory run for GLP materials.

Efficacy/Toxicology. Typically animal models are used in evaluating the safety, immunogenicity, and protection of vaccines. A small animal model is crucial for initial studies, due to the large number of animals required to show statistical relevance. Also, the mouse model has been shown in previous studies as an efficient means for testing EEEV protection that is similar to the protection anticipated in humans. Vaccines will typically be evaluated in two different animal models: mice and hamsters. The pathogenesis differs slightly in these animals, with mice exhibiting a more typical alphaviral encephalitis with neuronal death and perivascular infiltration, and hamsters showing more vascular lesions and multi-organ involvement characteristic of human EEE. Intraperitoneal challenges are used to maximize mortality and demonstrate strong efficacy. Alternatively, aerosol and mosquito challenges can be used to assess protection against the most common biowarfare and natural infection routes.

Vaccination of animals: In certain aspects of the invention intramuscular and intranasal administration will be used; the latter route was shown to be efficacious in a VEEV-adenoviral vaccine (Phillpotts et al., 2005).

Typically, adenovirus will be administered at a concentration ranging from 1×10⁹ to 1×10¹¹ vp. The efficacy and toxicology of the vaccines will first be evaluated in a mouse animal model. In general, challenge of vaccinated animals will be done with 100 LD₅₀ of EEEV strain FL93 obtained by rescue of an infectious cDNA clone. Linearized plasmid DNA will be transcribed in vitro and electroporated into BHK cells using standard methods. Virus has been rescued from this clone and its wild-type phenotype confirmed. Virus stocks can been titrated in adult (9-week-old) NIH Swiss mice to determine LD₅₀ doses via the subcutaneous (sc) and intraperitoneal (ip) routes. Mouse sc LD₅₀ values correspond to about 4 Log₁₀ PFU. Cohorts of 10 four-week-old Swiss NIH mice can be vaccinated with adenovirus, for example, at 1×10⁹, 5×10⁹, or 1×10¹¹ PFU. Animals can be bled retroorbitally (100 μl volume) and challenged with 10⁶ PFU (>100 LD₅₀) of EEEV via the ip route. Sham-vaccinated (diluent only) animals serve as controls.

Mortality will be monitored daily, and then surviving animals can be bled and sacrificed. A power analysis (Fisher's exact test) indicates initially that with >90% mortality in normal mice with this challenge dose a ≧80% protection level can be detected (cohort sizes of 10 mice (p<0.05, power=0.90)). Mortality rates can be compared between each vaccine and control cohort to assess protection, and pre- and post-challenge antibody titers can be determined using standard methods (Beaty et al., 1989). Mean survival times may also be compared, and viremia titers may also be measured and compared. Typically, if pre-challenge neutralizing antibodies are not detected, western blots and purified EEEV can be used to determine if the sera are reactive against organisms or proteins targeted by the vaccine. PRNT titers against both North and South American antigenic variants [EEEV strains C-49, BeAn5122 and BeAr436087, representing subtypes Il-IV (Brault et al., 1999), each of which is incorporated herein by reference in its entirety] may be monitored to assess protective effects of the inventive vaccines against other strains of organisms, e.g., all EEEV strains. Broad effects would be especially useful in an equine vaccine situations because South American strains also cause equine encephalitis.

Mosquito bite challenge. The dose delivered by infected mosquitoes typically cannot be regulated, but recent work with VEEV indicates that about 1-3 Log₁₀ PFU are inoculated in vivo. The methods include obtaining a first filial generation (FI) adult female Aedes taeniorhynchus females, which are efficient vectors of EEEV, and infecting them intrathoracically with 10³ PFU, incubated 5 days, then placed into small cartons with polyester mesh lids. Vaccinated or sham-vaccinated mice can be anesthetized with pentobarbitol and placed on the lid of a cage containing one infectious mosquito. Following engorgement, mice will be assessed for survival. After engorgement, mosquito saliva will be collected in capillary tubes filled with oil to ensure infectiousness of the bite. For these studies, typically two vaccine doses are used (1×10⁹ or 1×10¹⁰ pfu).

Aerosol challenge. Aerosol challenge can be performed in a BSL-3 aerobiology lab using a “nose only” exposure unit and 1-5 μm droplet sizes. LD₅₀ values can be determined to optimize the challenge dose. Vaccinated and sham-vaccinated mice can be exposed for 30 min or more with 100 LD₅₀ and then held for survival and disease assessment. Because protection against aerosol challenge may depend more on mucosal immunity that mosquito or ip challenge, bronchial lavage will be performed on some mice after vaccination to assess IgA and IgG levels using ELISA. For these studies, a vaccine dose of 5×10⁹ or 1×10¹⁰ pfu are used.

### GMP Production of a Vaccine.

Document preparation, review, and GMP production includes various activities in order to prepare for the cGMP manufacture of an adenoviral vaccine. A single batch of vaccine is typically produced. A vial or vials of cells from a Cell Bank can be thawed and expanded. The bioreactors or other production means will be processed using established procedures and infected with a virus bank. The infected reactor will then be harvested and processed.

Dedicated equipment is typically used throughout the processes including all ultrafiltration equipment and chromatography resin, with the exception of the chromatography system. The chromatography system can be cleaned and have all elastomeric components changed out prior to the manufacturing process.

Master Cell Bank (MCB) and Master Virus Bank (MVB) generation and characterization: During cell expansion procedure for cGMP manufacture of a vaccine, a MCB can be vialed and frozen. The starting material for the cell expansion is typically the tested Cell Bank that is established during feasibility studies. The MCB size will be subject to the cell growth of a particular batch, but will be targeted at providing approximately 100 vials at 2×10⁷ cells/vial. A portion of the final product of the cGMP production run is typically vialed and labeled to establish a MVB. As with the MCB, the material used to infect will be a tested Virus Bank established as part of a feasibility study. The MVB size will be subject to the cell growth of a particular batch, but will be targeted at providing approximately 200 vials at 2×10¹² vp/vial. Characterization for the MCB and MVB typically follow established standard testing.

Fill: A vaccine is typically vialed, inspected, and labeled using an appropriate label. Due to the unique fill requirements for a vaccine, a fill qualification will be required for verification. Placebo vials may also be filled in preparation for a clinical trial.

Evaluation Of The Candidate In A Second Animal Model. Testing for efficacy in humans is generally not possible short of clinical trials. Accordingly, the surrogate Golden Syrian hamster model can be used in conjunction with mouse model to assess vaccine candidates. Based on previous studies the Syrian hamster model has been shown to be an effective means for testing EEEV due to its 100% mortality upon exposure and histopathological disease that closely resembles human EEE.

For vaccines that show significant protection in murine studies additional evaluation is typically conducted using a hamster model. Hamsters may be challenged using mosquito inoculation and aerosol exposure.

### Example 2

### Yersinia pestis

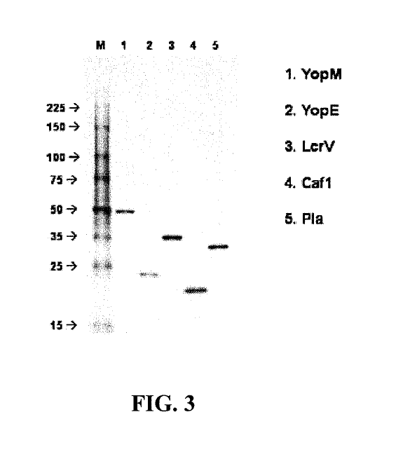

Purified recombinant antigens of Y. pestis. Several immunodominant antigens of Y. pestis were purified after cloning and expression in E. coli. Each antigen was isolated by Ni²⁺-affinity chromatography as N-terminal His-Tag labeled protein. Two of them (Caf1 and LcrV) representing known protective antigens of Y. pestis are employed as control peptides to evaluate the degree of protection provided in an animal model and their ability to induce humoral immune response by using adenoviral vector. FIG. 3 shows the quality of various Y. pestis CO92 purified antigens as estimated by SDS-PAGE and silver staining. Recently, the inventors also cloned and expressed YscF by similar methods and are in the process of pilot scale purification.

Passive immunity to Y. pestis KIM. Proteins consisting of the immunoglobulin G-binding domain of staphylococcal protein A (PA) and LcrV of Y. pestis (PAV) have been produced (Motin et al., 1994). The proteins PA and PAV were purified and used to immunize rabbits. Rabbit polyclonal gamma globulin directed against PAV provided excellent passive immunity against 10 median lethal doses (MLD) of Y. pestis (P<0.005). In this determination (Table 2), lethality to untreated mice was absolute and occurred rapidly in a pattern similar to those observed for control animals treated with purified normal IgG or IgG generated to PA alone.

TABLE 2
Ability of IgG isolated from rabbit sera raised against different antigens to provide
passive immunity against intravenous challenge with 10 MLD of Y. pestis

| | | Number of Mice surviving on day after infection | | | | | | | | | | | | | | | | Dead/ | |
|---|---|---|---|---|---|---|---|---|---|---|---|---|---|---|---|---|---|---|---|
| Organism | Source IgG | Dy 0 | 1 | 2 | 3 | 4 | 5 | 6 | 7 | 8 | 9 | 10 | 11 | 12 | 13 | 14 | 21 | total | P value |
| Y. pestis | Normal | 10 | 10 | 10 | 10 | 6 | 4 | 1 | — | — | — | — | — | — | — | — | — | 10/10 | |
| KIM | Anti-native | 10 | 10 | 10 | 10 | 10 | 10 | 10 | 10 | 10 | 10 | 10 | 10 | 10 | 10 | 10 | 10 | 0/10 | <0.005 |
| | V antigen | | | | | | | | | | | | | | | | | | |
| | Anti-recombinant | 10 | 10 | 10 | 10 | 8 | 6 | 6 | 6 | 6 | 6 | 6 | 6 | 6 | 6 | 6 | 6 | 4/10 | <0.01 |
| | V antigen | | | | | | | | | | | | | | | | | | |
| | Anti-PAV | 10 | 10 | 10 | 10 | 10 | 10 | 10 | 10 | 10 | 9 | 9 | 9 | 9 | 9 | 9 | | 1/10 | <0.01 |
| | Anti-truncated | 10 | 10 | 10 | 10 | 3 | 1 | — | — | — | — | — | — | — | — | — | — | 10/10 | NS |
| | protein A | | | | | | | | | | | | | | | | | | |

TABLE 3
Ability of Vh to protect mice against intravenous challenge with Y. pestis (Lcr+)

No.

| injected | Bacterial | | Number of Mice surviving on post-infection day(s) | | | | | | | | | | | | | | |
|---|---|---|---|---|---|---|---|---|---|---|---|---|---|---|---|---|---|
| bacteria | MLD | Immunogen | Dy 0 | 1 | 2 | 3 | 4 | 5 | 6 | 7 | 8 | 9 | 10 | 11 | 12 | 13 | 14-21 |
| 10² | 10¹ | None | 9 | 9 | 9 | 8 | 7 | 6 | 3 | 2 | 2 | 2 | 1 | 1 | 0 | 0 | 0 |
| 10³ | 10² | None | 10 | 10 | 10 | 8 | 6 | 0 | 0 | 0 | 0 | 0 | 0 | 0 | 0 | 0 | 0 |
| 10⁴ | 10³ | None | 10 | 10 | 10 | 9 | 4 | 3 | 2 | 0 | 0 | 0 | 0 | 0 | 0 | 0 | 0 |
| 10² | 10¹ | Vₕ | 10 | 10 | 10 | 10 | 10 | 10 | 10 | 10 | 10 | 10 | 10 | 10 | 10 | 10 | 10 |
| 10³ | 10² | Vₕ | 10 | 10 | 10 | 10 | 9 | 9 | 9 | 9 | 9 | 9 | 9 | 9 | 9 | 9 | 9 |
| 10⁴ | 10³ | Vₕ | 10 | 10 | 10 | 10 | 9 | 9 | 9 | 9 | 9 | 9 | 9 | 9 | 9 | 9 | 9 |
| 10⁵ | 10⁴ | Vₕ | 10 | 10 | 10 | 10 | 10 | 10 | 10 | 10 | 10 | 10 | 10 | 10 | 10 | 10 | 10 |
| 10⁶ | 10⁵ | Vₕ | 10 | 10 | 10 | 9 | 9 | 9 | 9 | 9 | 9 | 9 | 9 | 9 | 9 | 9 | 9 |
| 5 × 10⁶ | 5 × 10⁵ | Vₕ | 10 | 10 | 10 | 10 | 10 | 10 | 10 | 10 | 10 | 10 | 10 | 10 | 10 | 10 | 10 |
| 10⁷ | 10⁶ | Vₕ | 10 | 10 | 10 | 10 | 8 | 7 | 7 | 7 | 7 | 6 | 6 | 6 | 6 | 6 | 6 |

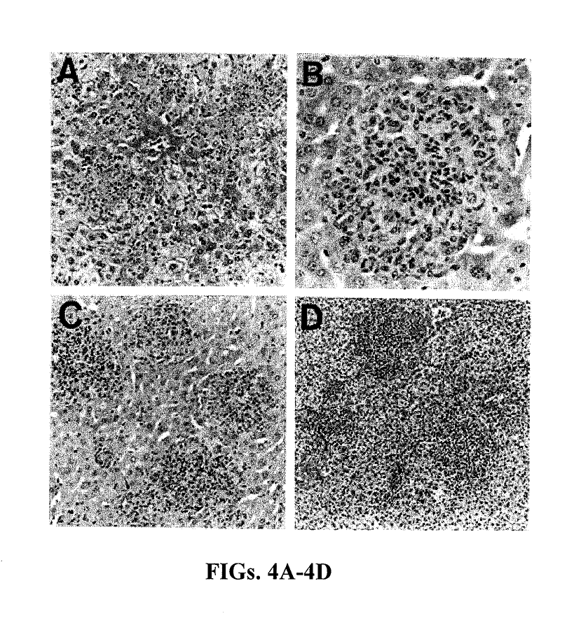

Histopathological changes caused by Y. pestis. Intravenous injection of Y. pestis KIM strain into nonimmunized control mice or those actively immunized with PA resulted in severe damage to the liver (FIG. 4A) and spleen (not illustrated) by postinfection day 3. Infiltration of inflammatory cells to these necrotic foci was never observed, underscoring the acute nature of the infection. In contrast, lesions formed in organs of mice actively immunized with PAV attracted massive numbers of neutrophils and mononuclear cells, resulting in their conversion to protective granulomas by postinfection day 3 (FIG. 4C). These granulomas closely resembled those formed in response to injection of avirulent Y. pestis cells (Lcr−) into normal control mice (FIG. 4B) or of virulent Y. pestis cells (Lcr+) into mice passively immunized with anti-PAV (FIG. 4D) (Nakajima et al., 1995).

Active immunity against plague. Mice were immunized with His-tagged LcrV (Vh) according to the protocol developed for PAV, and the resulting concentrations of antibodies were determined by ELISA. Results showed that titers after injection of Vh were about 100 times greater than those previously determined for mice actively immunized with PAV. Control mice previously injected with adjuvant alone and mice injected with Vh emulsified in adjuvant were challenged intravenously with Lcr+ cells of Y. pestis KIM. As shown in Table 3, the 50% lethal dose for immunized mice was >10⁷ organisms (Motin et al., 1996).

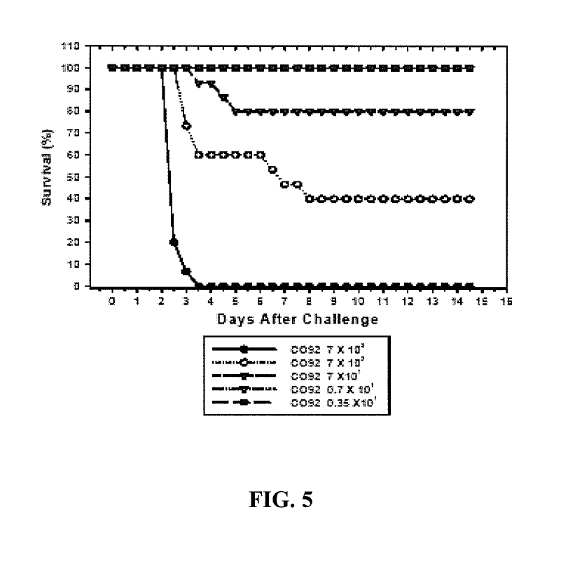

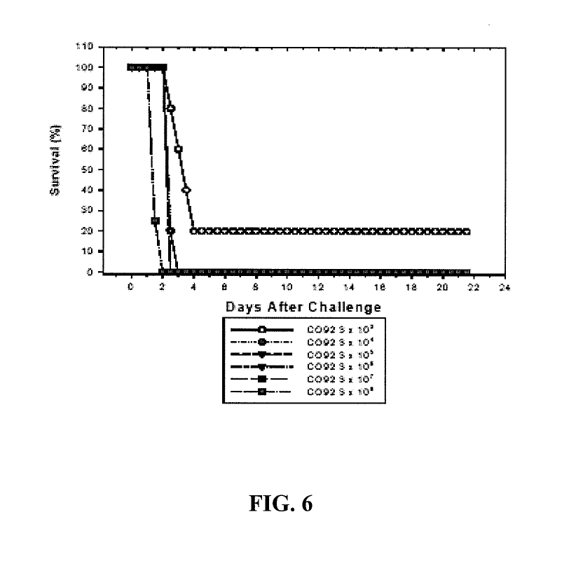

LD₅₀ dose of a highly virulent Y. pestis CO92 strain via the intranasal route. The inventors have determined the LD₅₀ dose of Y. pestis CO92 in mice for the first time by the intranasal route. Since the ultimate goal is to use aerosolized bacteria for infection, the inventors preferred to infect animals via the intranasal route which closely mimics aerosol route of infection. In the first study, doses ranging from 3×10³ to 3×10⁸ cfu were used. Five mice/dose were used in this study, and as can be noted from FIG. 5, all of the animals infected with doses ranging from 3×10⁴ to 3×10⁸ cfu died within 3 days of inoculation. The mortality rate was 80% at the dose of 3×10³ cfu. In subsequent study, animals were infected with bacterial doses ranging from 0.35×10¹ to 7×10³ cfu. In this study, 15 mice/dose were used. As noted from FIG. 6, all of the animals infected with Y. pestis CO92 at the dose of 7×10³ cfu died within 4 days. The mortality rate was 60% by day 8 at the dose of 7×10² cfu. A 20% mortality was noted at the dose of 7×10¹ cfu. Based on the method of Reed and Munch for determining LD₅₀, the LD₅₀ dose for Y. pestis CO92 was calculated to be 340 bacteria.

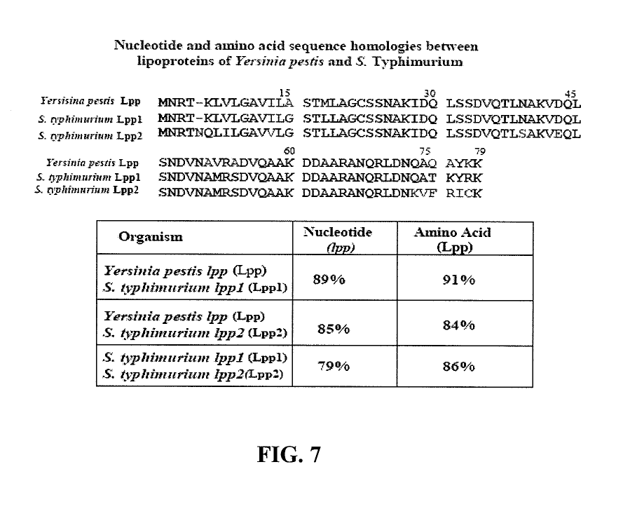

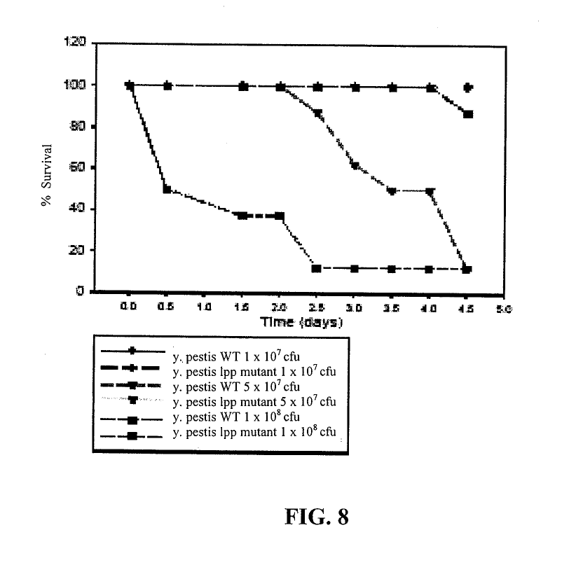

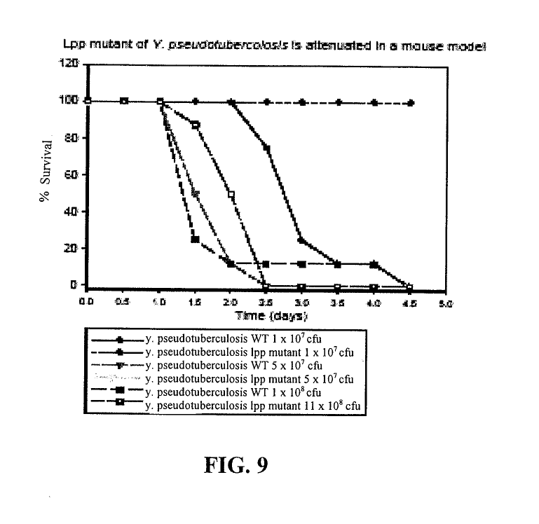

Identification of new antigens to attenuate yersiniae. The inventors have been actively involved in identifying new antigens that could be either deleted to attenuate yersiniae or in identifying those antigens that could elicit immune response in the host. The inventors have recently shown that murein (Braun) lipoprotein, which constitutes one of the major components of bacterial outer membrane in the family enterobacteriaceae, is critical in inducing inflammatory response in the host. Using Salmonella Typhimurium as a model system, that deletion of the lipoprotein (lpp) gene was shown attenuated the bacterium, and mice immunized with such a mutant were protected from lethal challenge dose of the wild type (WT) bacterium (Sha et al., 2004; Fadl et al., 2005a; Fadl et al., 2005b). The genome sequence analysis of Y. pestis (both KIM and CO92) and Y. pseudotuberculosis indicated presence of a copy of the 1 pp gene with 85-90% homology with the 1 pp genes of S. Typhimurium (Salmonella harbors two copies of the 1 pp gene) (FIG. 7). Therefore, by using marker exchange mutagenesis employing a suicide vector, the gene encoding Lpp was deleted from both Y. pestis KIM and Y. pseudotuberculosis. These mutants then were tested for their attenuation in a mouse model after intraperitoneal inoculation. As noted in FIG. 8, approximately 90% of the animals infected with the WT bacterium died at the doses of 1×10⁸ and 5×10⁷ cfu after 5 days of infection. However, majority of the animals survived these doses when the 1 pp gene was deleted. Based on studies assessing the LD₅₀ dose of Y. pestis KIM by intranasal route the inventors noted that the LD₅₀ fell within the range of 1×10⁷ to 5×10⁷ cfu. Doses of 1×10⁷ to 1×10⁸ cfu were selected for further study. Lpp deficient Y. pseudotuberculosis was shown to be attenuated when injected intraperitoneally (FIG. 9). As can be noted from this figure, all of the animals infected with the WT bacterium at doses of 1×10⁷ to 1×10⁸ cfu died within 5 days. Also higher doses (1×10⁸ and 5×10⁷ cfu) of the Lpp mutant killed all of the animals; however, the mean death time for the Lpp mutant appeared longer compared to the animals infected with the WT bacterium. More importantly, at a dose of 1×10⁷ cfu, although all of the animals infected with the WT bacterium died, none died with the Lpp mutant. These studies are ongoing and the animals will be observed for a period of minimum of 21 days. The inventors plan to repeat these studies with lower doses of Y. pseudotuberculosis and also use intranasal route of inoculation.

Vaccine Construction. Typically, constructs are generated by inserting a target gene (antigenic determinant expression cassette) into a replication-defective or replication-competent adenovirus backbone via a shuttle vector. The shuttle vector is then processed using standard recombination protocol to generate the adenovirus-plague vector. Briefly, 293INT cells will be cultured in a 6-well plate or other equivalent format and infected with a construct of the invention. Cells infected either with the vector containing the lcrV, caf1, or the yscF gene alone or in combination lcrV, caf1, and yscF will be further cultured and monitored for the development of plaques. The infection and plaque formation cycle is estimated to take 7 to 10 days, and a single round of plaque purification will be used.

The expression design relies on the previous finding that deletion of the first 67 amino acid (a.a.) residues of the N-terminus of LcrV does not significantly influence the protective properties of this antigen (Motin et al., 1994; Motin et al., 1996). Although one of the epitopes located in this area might contribute to the protection (Hill et al., 1997), this region is involved in the immunosuppressive effect mediated by LcrV likely due to IL-10 induction via Toll-like receptor-2 (Sing et al., 2002; Sing et al., 2005). Therefore, deletion of this region will eliminate the controversy of whether it is safe or not to use the intact LcrV in vaccine formulations (Overheim et al., 2005). Thus, the size of LcrV expressed in adenovirus vector will be 259 amino acid residues (SEQ ID NO:1 encoding SEQ ID NO:2), and the size of Caf1 (SEQ ID NO:3 encoding SEQ ID NO:4) and YscF (SEQ ID NO:5 encoding SEQ ID NO:6) antigens will be 170 amino acids (mature form) and 87 amino acid residues, respectively. The combined genetic information required for the expression of all three antigens in tri-valent vaccine candidate is within the capacity of various adenoviral vectors (6 kb or more). Each antigen in the tri-valent vaccine will be expressed from its own controlled element within the same construct.

Purification, Characterization, and Sequencing. After formation of plaques, 5 to 10 plaques are chosen. Their DNA will be purified from the progeny virus, and analyzed by the PCR assay. A selected number of these plaques will be identified as non-recombinant. Of this group of non-recombinants, up to three plaques will be then further amplified in 293INT cells and again analyzed by the PCR assay to confirm that no recombinants are present. One of these cultures will then be chosen as a master stock, to be used for further work and to generate the Virus Bank. Characterization and sequencing will be performed to confirm the composition of the master stocks. Characterization activities include, but are not limited to, the detection of mycoplasma, sterility, particle counts, and infectivity. Characterization will be performed on 1) the initial construct, 2) the cell bank (RCB), 3) the virus bank (RVB). In parallel to the characterization, nucleic acid analysis assays will be developed for the detection of the correct sequence for the adenoviral-plague vectors, e.g., PCR assays. Sequencing will consist of approximately the length of any heterologous DNA (and may include all or part of the adenoviral genome) and can be 0.5, 1, 2, 4, 6, 10, 20, 30 or more kb to verify adenoviral constructs.

Feasibility Studies. The feasibility study will determine the compatibility of the manufacturing process with the needs of the plague vaccine. Various parameters and characterizations will be documented including, but not limited to 1) multiplicity of Infection (MOI) studies, 2) effect of cell density at time of infection, 3) effect of infection temperature, and 4) confirmation of activity with 293INT cells.

GLP production of the vaccine candidates for animal studies. Small production scale process will be performed under GLP to test the suitability of the products for large scale manufacture. The step is considered in justification of large scale production and clinical development of any product. In this study, the production of GLP-grade material will serve two purposes: 1) confirmation of scalability to GMP and commercial scale quantities, and 2) supply material for subsequent animal studies.

GLP manufacturing procedures will be performed during preparation of the adenoviral-LcrV and other adenoviral vaccines necessary for the following animal studies. Four exemplary products include, but is not limited to 1) Ad-LcrV; 2) Ad-Caf1; 3) Ad-YscF; and 4) Ad-LcrV+Caf1+YscF.

Small Animal model efficacy/toxicology. Animal models will be instrumental in evaluating the safety, immunogenicity, and protection of the vaccines. A small animal model is typically used for initial studies, due to the large number of animals required to show statistical relevance. Also, the mouse model has been shown in previous studies as an efficient means for testing plague protection. Upon completion of this part of the project, the vaccines will be further evaluated using a second animal model (guinea pig).

Vaccination and Serology. Vaccines will be assessed using two different routes of administration: intramuscular and intranasal. The adenovirus will be administered at a concentration ranging from 1×10⁹ to 1×10¹⁰ vp. Studies will be conducted in compliance with the Animal Welfare Act and other federal regulations and will adhered to principles stated in the Guide for the Care and Use of Laboratory Animals, National Research Council, 1996. Previously established mouse models for bubonic and pneumonic plague will be used to compare adenovirus plague vaccine formulations (LcrV, Caf1, and YscF alone and a tri-valent vaccine LcrV-Caf1-YscF) with the control subunit vaccine LcrV alone or the cocktail of purified antigens LcrV+Caf1+YscF. The control vaccines will be administered by intramuscular injection into the hind leg as a single dose amount (0.6 nmol) per delivered protein antigen preadsorbed to adjuvant (Alhydrogel). Mice will be bled weekly and the antigen-specific antibody titer will be determined by an immuno-ferment assay including Ig-subclass titers. It is anticipate that the protective properties of the adenovirus plague vaccines will correlate with the antigen-specific titers. In pilot studies, the inventors will determine the regime of vaccination that provides the highest antigen-specific titers, and then use this regime in protection studies.

Protocol I:

Groups: Groups will include 1. Control (adenoviral vector), 2. Adenoviral LcrV, 3. Adenoviral tri-valent, 4. Subunit LcrV, 5. Subunit cocktail LcrV+Caf1+YscF.

Routes: Routes of vaccination include IM (all groups) and Intranasal (groups 1-3)

Immunization: Primary Immunization (day 0) will include—Adenoviral Vaccines (1×10⁹ to 1×10¹⁰) or Subunit Vaccines (0.6 nmol per antigen per mouse)

Bleeding: Bleeding will be conducted on day −1 then weekly for 10 weeks.

Boosting: Boosting will be conducted as two identical sets of groups 1-5 will be tested: one set will receive only primary immunization and another set will receive an identical boost on week 4.

Monitoring: Animals will be monitored for signs of animal illness due to possible vaccine toxicity and for antigen-specific antibody titers including Ig-subclass.

Each group will contain 10 mice. Intramuscular injection: 5 groups×10 mice=50 mice; intranasal injection: 3 groups×10 mice=30 mice. 80 mice per immunization schedule will be used. Since two schedules of immunization will be utilized (with and without boost), the total number of mice is 160 and are to be studied for 10 weeks. A second boost on week 8 might be needed in case the antibody titers are low in adenoviral vaccine groups. In this case monitoring of animals will be extended beyond 10 weeks, likely to 12-14 weeks.

Statistical calculations. Vaccine efficacy will be estimated directly from animal survival as analyzed by life table techniques (SAS Institute Inc., Cary, N.C.) to determine the mean time of survival (MST). Five doses of fully virulent Y. pestis strain CO92, ranging from 10³ to 10⁷ median lethal doses (MLD), will be utilized for the protection studies and administered either subcutaneously for bubonic plague model or intranasally corresponding to a pneumonic plague model. The time of challenge will be chosen based on the results of the immunization efficiency study when the specific antibody titers reach a plateau.

The animals will be monitored for four weeks following the challenge. Clinical observations and mortality will be recorded during this four-week period. Any mouse found dead or moribund will be sacrificed, and a necropsy will be performed. The bacterial load in spleens, livers, and lungs will be determined by plating organ homogenates in 10-fold dilutions.

Protocol II:

Groups: Groups will include 1. Control (adenoviral vector), 2. Adenoviral LcrV, 3. Adenoviral tri-valent, 4. Subunit LcrV, and 5. Subunit cocktail LcrV+Caf1+YscF.

Routes: Routes of vaccination include IM (all groups) and intranasal (groups 1-3)

Immunization: determined from protocol I.

Challenge: Subcutaneously or intranasally 10³ to 10⁷ MLD (five doses per each route, 10-fold dilutions).

Time Frame: Monitor for 28 days post challenge.

Assessment: Mice assessed for clinical illness, morbidity and mortality, histopathology, and bacterial load in organs.

Each group will contain 10 mice. Intramuscular injection: 5 groups×10 mice=50 mice; intranasal injection: 3 groups×10 mice=30 mice. Therefore, 80 mice per immunization schedule will be used. Since two routes of injection with Y. pestis will be utilized, a total number of mice will be 160 per dose. Five doses will be evaluated bringing total number of mice to 800. The entire experiment (or it parts) will be repeated at least once, so the final number of mice required will be 1,600.

Aerosol challenge remains the gold standard as a model for pneumonic plague, although intranasal administration of Y. pestis is likely a good substitute route for initial screening of the protective efficacy. Taking into account the importance of the evaluation of the vaccine against pneumonic plague, the inventors plan to conduct an aerosol study of protection for adenoviral vaccine candidates. Two aerosol doses will be chosen based on the results of protection against intranasal inoculation for each route of immunization. The animals will be monitored for four weeks following the challenge. Clinical observations and mortality will be recorded during this period. Any mouse found dead or moribund will be sacrificed, and a necropsy will be performed. The bacterial load in spleens, livers, and lungs will be determined by plating organ homogenates in 10-fold dilutions.

Protocol III:

Groups: Groups will include 1. Control (adenoviral vector), 2. Adenoviral LcrV, 3. Adenoviral tri-valent, 4. Subunit LcrV, and 5. Subunit cocktail LcrV+Caf1+YscF.

Routes: Routes of vaccination include IM (all groups) and intranasal (groups 1-3)

Immunization: Schedule determined from protocol I and protocol II.

Challenge: Aerosol challenge using two doses selected based on results of protocol II.

Time Frame: Monitor for 28 days post challenge.

Assessment: Mice assessed for clinical illness, morbidity and mortality, histopathology, and bacterial load in organs.

Each group will contain 10 mice. Intramuscular injection: 5 groups×10 mice=50 mice; intranasal injection: 3 groups×10 mice=30 mice. Therefore, 80 mice per immunization schedule will be used. One route of injection with Y. pestis will be utilized (aerosol), and two doses will be used, resulting in a total 160 mice being used.

Evaluation of Vaccine in Second Animal Model

Due to the unique nature of these compositions, testing for efficacy in humans is not possible. Accordingly, the guinea pig model will be used as the second part of the two-animal model as dictated for high priority agents. Guinea pigs have been traditionally used as an animal model for plague infection because they are susceptible to the disease. Guinea pigs that are infected via a flea bite tend to exhibit a red areola around the wound with the development of a red papule within days. Similar lesions have been observed following intradermal but not subcutaneous injection of the plague bacterium. Following the development of a papule, the draining lymph node swells, and this is followed by septicemia and death within 2 weeks. On post-mortem examination of animals that died after subcutaneous injection of Y. pestis, it was found that the guinea pigs had lesions disseminated on most organs, which were also culture-positive for Y. pestis. Importantly there was clear infection in the lungs of these animals indicating that subcutaneous injection had led to secondary pneumonic infection (Jones et al., 2003).

Vaccines showing significant protection in initial murine studies will be assessed further using the guinea pig model. It is expected that a high-titer antibody response to the adenoviral plague vaccine candidates would be important for protection in the guinea pig. Current experience in using guinea pigs for evaluation of the anti-plague subunit vaccine indicated that the response to Caf1 took longer to rise than the response to LcrV, being undetectable prior to boosting (Jones et al., 2003). Therefore, the optimal vaccination schedule for adenoviral plague vaccine will be determined prior to challenge studies, similar to that performed in mice for the Protocol I. The inventors will use only intramuscular route of immunization, and subcutaneous and aerosol routes of administration of Y. pestis

Protocol IV:

Groups: Groups will include 1. Control (adenoviral vector), 2. Adenoviral LcrV, 3. Adenoviral tri-valent, 4. Subunit LcrV, and 5. Subunit cocktail LcrV+Caf1+YscF.

Routes: Routes of vaccination include IM (all groups).

Immunization: Primary Immunization (day 0) will include—Adenoviral Vaccines (1×10⁹ to 1×10¹⁰) or Subunit Vaccines (2 nmol per antigen per guinea pig)

Bleeding: Bleeding will be conducted on day −1 then weekly for 12 weeks.

Boosting: Boosting will be conducted as two identical sets of groups 1-5 will be tested: one set will receive only primary immunization and another set will receive an identical boost on week 4.

Monitoring: Animals will be monitored for signs of animal illness due to possible vaccine toxicity and for antigen-specific antibody titers including Ig-subclass.

Each group will contain 6 animals as chosen in another study of evaluation of subunit anti-plague vaccine in guinea pigs (Jones et al, 2003). Intramuscular injection: 5 groups×5 guinea pigs=30 animals. Since two schedules of immunization will be utilized (with and without boost), a total number of guinea pigs is 60 which will be studied for 12 weeks. A second boost on week 8 might be needed in case the antibody titers are low in adenoviral vaccine groups. In this case monitoring of animals will be extended beyond 12 weeks, likely to 14-16 weeks.

Statistical calculations. Vaccine efficacy will be estimated directly from animal survival as analyzed by life tables techniques (SAS Institute Inc., Cary, N.C.) to determine the mean time of survival (MST). Two doses of a fully virulent Y. pestis strain CO92, corresponding to the protective doses determined in the study involved mice, will be utilized for the protection studies administered either subcutaneously or by aerosol corresponding to bubonic and pneumonic plague models, respectively. The time of challenge will be chosen based on the results of the immunization efficiency study when the specific antibody titers reach a plateau.

The animals will be monitored for the period of four weeks following the challenge. Clinical observations and mortality will be recorded during this four-week period. Any guinea pig found dead or moribund will be sacrificed and a necropsy will be performed. The bacterial load in spleens, livers, and lungs will be determined by plating organ homogenates in 10-fold dilutions.

Protocol V:

Groups: Groups will include 1. Control (adenoviral vector), 2. Adenoviral LcrV, 3. Adenoviral tri-valent, 4. Subunit LcrV, and 5. Subunit cocktail LcrV+Caf1+YscF.

Routes: Routes of vaccination include IM (all groups)

Immunization: determined from protocol IV.

Challenge: Subcutaneously or aerosol (two doses per each route).

Time Frame: Monitor for 28 days post challenge.

Assessment: Guinea pigs assessed for clinical illness, morbidity and mortality, histopathology, and bacterial load in organs.

Each group will contain 6 animals. Intramuscular injection: 5 groups×6 guinea pigs=30 animals. Since two routes of injection with Y. pestis will be utilized (subcutaneous and aerosol), the total number of guinea pigs will be 60 per dose. Two doses will be evaluated bringing the total number of guinea pigs to 120.

Overall, upon completion of these studies the vaccine will have been fully evaluated in 2 relevant animal models. The inventors contemplate that this project will produce a safe and efficacious plague vaccine in preparation for GMP production and Phase I human testing.

Methods

Y. pestis challenge by aerosolization and subcutaneous injection. Fully virulent Y. pestis strain CO92 which is routinely used in UTMB laboratories (Drs. Motin and Chopra) will be employed to test the efficacy of treatment of plague infection. For the past decade, this strain, isolated from a human case of pneumonic plague, has become a reference strain in the United States. The LD₅₀ (lethal dose for 50% of mice) of this strain is well established for various routes of infection. The LD₅₀ doses for the subcutaneous (s.c), intraperitoneal (i.p.), and aerosol routes are 1.9, 14, and 2.3×10⁴ colony forming units (CFU), respectively (Welkos et al., 1995; Welkos et al., 1997; Worsham et al., 1995). The inventor have determined the LD₅₀ dose by the intranasal route as 340 bacteria.

Bacterial cells will be prepared and used in challenge experiments as described previously by Anderson et al. (1996). Cultures will be grown in heart infusion broth (HIB; Difco Laboratories) at 28° C., harvested, washed, suspended in HIB, and adjusted to an A₆₂₀ of 1.0 (approximately 10⁹ CFU/ml). Animals will be challenged s.c. in the hind leg using different doses (10-fold diluted samples) of CO92 in 0.2 ml, and the exact number of cells inoculated will be determined by plating the bacterial suspension on tryptose blood agar base plates, TBA (Difco Laboratories). The LD₅₀ will be calculated by Probit analysis on data accumulated by the last day of observation (Wlekos and O′Brien, 1994. For aerosol infection, inhaled doses will be administered to animals by nose-only aerosol exposure (In-Tox Products, Moriarty, N. Mex.) in Aerosol Challenge Facility at UTMB. The aerosol will be generated by the Lovelace Nebulizer in particle sizes of 1.5-3 μm, and the aerosol stream will be maintained at 50-55% relative humidity and 22±3° C. Animals will be exposed to aerosol for 10 min. The aerosol concentration will be determined by plating dilutions of the sampled aerosol on TBA plates and counting the colonies. The inhaled dose (CFU/animal) will be estimated by using Guyton's formula (Guyton, 1947), followed by removing the lungs from control animals and plating their homogenates onto Congo Red agar.

Histopathology. Histopathology will be performed as described previously for animal models of bubonic and pneumonic plague (Byrne et al., 1998). Tissue samples of all major organs will be collected from approximately 50% of the dead (including the euthanatized) animals during all studies. These samples will be fixed in 10% neutral buffered formalin and then routinely processed, embedded in paraffin, and sectioned (5- to 6 μm-thick sections) for hematoxylin and eosin staining. In addition to looking for histopathological changes in tissues of vaccinated animals, the inventors will also look for localization of the pathogen using fluorescent antibody staining. Typically, the rehydrated sections will be counter-stained, rinsed, and covered with primary antibody (usually with polyclonal monospecific rabbit anti-capsular antiserum). Control slides containing the same tissues will be overlaid with antibody to a heterologous antigen. At the end of the one hour incubation, the slides will be rinsed with PBS and overlaid for one hour with the secondary antibody labeled with fluorescein isothiocyanate (FITC). At the end of this incubation, the slides will be rinsed and observed under a fluorescent microscope for Y. pestis-specific fluorescence.

IgG ELISA. Serum antibody titers will be determined by direct IgG enzyme-linked immunosorbent assay (ELISA). 96-well plates (Immulon, Dynatech Laboratories, Chantilly, Va.) will be coated with 0.1 μg of purified recombinant antigen in PBS (all antigens used in this study are soluble). After blocking of the wells with 5% skim milk, serum samples will be applied at a starting dilution of 1:100 and serially diluted twofold to 1:12,800 in a final volume of 100 μl per well. The plates will be incubated for 2 h at room temperature (RT), washed and then a 1:2,000 dilution of horseradish-conjugated goat anti-mouse or guinea pig IgG will be added at 100 μl per well. After 1 h incubation at RT, plates will be washed and 100 μl of 2,2′-azinobis(3-ethylbenzthiazolinesulfonic acid) (ABTS) two-component substrate system (Sigma) will be added. Following incubation at RT for 30 min, plates will be read at 405 nm. The endpoint titer will be determined by the highest test serum dilution with optical density at 405 nm (OD₄₀₅) of ≧0.20 after subtraction of the OD₄₀₅ for the blank wells with no antigen. The results are expressed as the geometric mean titer of the reciprocal endpoint dilution for three separate experiments. A negative result means the test serum was less than two fold higher than normal mouse serum tested against the same antigen.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. Aspects of one embodiment may be applied to other embodiments and vice versa. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Azimi 1997. Pediatr Infect Dis J 16:824-25.
Barber et al., 1978. Am J Vet Res 39:621-5.
Bartelloni et al. 1970. Am J Trop Med Hyg 19: 123-6.
Bastian et al. 1975. Am J Clin Pathol 64: 10-3.
Beaty et al. 1989. Arboviruses, p. 797-855. In N.J. Schmidt and R. W. Emmons (ed.), Diagnostic procedures for viral, rickettsial and chlamydial infections, 6th edition. American Public Health Association, Washington, D.C.
Brault et al. 1999. Am. J. Trop. Med. Hyg. 61 579-86.
Brault et al. 2002. J. Virol. 76:1718-30.
Charles et al. 1995. Virology 208:662-71.
Cheng et al. 1995. Cell 80:621-30.
Del Piero et al. 2001. Vet Pathol 38:451-6.
Deresiewicz et al. 1997. N Engl J Med 336:1867-74.
Dropulic et al. 1997. J Virol
Elvinger et al. 1994. J Am Vet Med Assoc
Farber et al. 1940. JAMA 114:1725-31.
Femster 1957. N Engl J Med 257:701-4.
Femster 1938. N Engl J Med 28:1403-10.
Franklin et al. 2002. Emerg Infect Dis 8:283-8.
Garen et al. 1999. Mod Pathol 12:646-52.
Getting 1941. N Engl J Med 224:999-1006.
Griffin 2001. Alphaviruses, p. 917-962. In D. M. Knipe and P. M. Howley (ed.), Fields'Virology, Fourth Edition. Lippincott, Williams and Wilkins, New York.
Guy et al. 1994. Avian Dis 38572-82.
Hodgson et al. 1999. Vaccine 17: 1 151-60.
Hunt et al. 1990. Virology 179:701-11.
Hunt and Roehrig. 1995. Vaccine 13:281-8.
Hunt et al. 1991. Virology 185281-90.
Jochim and Barber. 1974. J Am Vet Med Assoc
Johnson et al. 1991. Virology 185:840-2.
Johnson et al. 1990. Virology 177:676-83.
Jordan et al. 1965. Am J Trop Med Hyg 14:470-4.
Kinney et al. 1993. J. Virol. 67:1269-77.
Kuhn et al. 1990. J. Virol. 64:1465-76.
Lescar et al. 2001. Cell 105:137-48.
Liu et al. 1970. J Infect Dis 12253-63.
Ludwig et al. 1996. J Virol 705592-9.
Maire et al. 1970. Am J Trop Med Hyg 19:119-22.
Mathews and Roehrig. 1982. Journal of Immunology 129:2763-7.
Nathanson et al. 1969. J Comp Pathol 79:109-15.
Niesters and Strauss. 1990. J Virol 64:1639-47.
Paessler et al. 2004. J Infect Dis 189:2072-6.
Paredes et al. 2001. J. Virol.
Paredes et al. 1993. Proc Natl Acad Sci USA
Pedersen 1976. J Clin Microbiol 3: 1 13-8.
Pereboev et al. 1993. Vopr Virusol 38: 1 17-22.
Pereboev et al. 1996. Arch Virol 141:2191-205.
Phillpotts et al. 2005. Vaccine 23:1615-23.
Pletnev et al. 2001. Cell 105127-36.
Roehrig et al. 1988. Virology 16566-73.
Roehrig and Mathews. 1985. Virology 142:347-56.
Rozdilsky et al. 1968. Can Med Assoc J 98:79-86.
Schoepp et al. 2002. Virology 302:299-309.
Scott and Weaver. 1989. Adv. Virus Res. 37:277-328.
Snoeyenbos et al. 1978. Avian Dis 22:386-90.
Strizki and Repik. 1995. Am J Trop Med Hyg 53564-70.
Tsai et al. 2002. Alphaviruses, p. 1177-1210. In D. D. Richman, R. J. Whitley, and F. G. Hayden (ed.), Clinical Virology. ASM Press, Washington, D.C.
Tucker and Griffin. 1991. J Virol 65:1551-7.
U.S. Department of Health and Human Services. 1999. Biosafety in Microbiological and Biomedical Laboratories, 4th edition. U.S. Government Printing Office, Washington, D.C.
Villari et al. 1995. Am J Trop Med Hyg 52:8-13.
Vogel et al. 2005. Am J Pathol 166:159-71.
Walder et al. 1980. Differentiation markers of eastern equine encephalitis (EEE) viruses and virulence, p. 237-250. In J. Vesenjak-Hirjan (ed.), Arboviruses in the Mediterranean Countires. Gustav Fischer Verlag, Stuttgart.
Wang et al., J Clin Microbiol. 44(11): 4000-4008, 2006.
Weaver 2001. Eastern equine encephalitis, p. 151-159. In M. W. Service (ed.), The Encyclopedia of Arthropod-transmitted Infections. CAB International, Wallingford, UK.
Weaver et al. 1999. Am. J. Trop. Med. Hyg. 60:441-8.
Woodward et al. 1991. J Gen Virol 72:2431-5
Boyer et al. 2005. Hum Gene Ther. 16(2): 157-68
Perry and Fetherston. Clin. Microbiol. Rev. 1997; 10:35-66
Cornelis and Van Gijsegem Annu. Rev. Microbiol. 2000; 54:735-774
Brubaker Infect Immun 2003; 71:3673-3681.
Une et al. Contrib. Microbiol. Immunol. 1986; 9:179-185
Titball and Williamson Vaccine 2001; 19:4175-4184.
Baker et al. J. Immunol. 1952; 68:131-145
Cornelis J. Cell Biol 2002; 158:401-408
Motin et al. Infect. Immun. 1994; 62:4192-4201
Nakajima et al. Infect. Immun. 1995; 63:3021-3029
Motin et al. Infect. Immun. 1996; 64:4313-4318
Oyston et al. Infect Immun 1995; 63:563-568.
Leary et al. Microb Pathog 1997; 23:167-179.
Garmory et al. Vaccine 2003; 21:3051-3057.
Williamson et al. Clin. Exp. Immunol. 1999; 116:107-114
Elvin and Williamson Microb Pathog 2000; 29:223-230.
Andrews et al. Infect. Immun. 1999; 67:1533-1537
Hoiczyk and Blobel Proc. Natl. Acad. Sci. U.S.A. 2001; 98:4669-4674
Matson et al. BMC Microbiol 2005; 5:38.
Swietnicki et al. Protein Expr Purif 2005; 42:166-172.
Anderson et al. Infect. Immun. 1996; 64:4580-4585
Davis et al. Arch. Pathol. Lab. Med. 1996; 120:156-163
Motin In: Cold Spring Harbor Symposium on Molecular Approaches to the Control of Infectious Diseases. Cold Spring Harbor, N.Y.: Cold Spring Harbor Laboratory, 1996
Sha et al. Infect Immun 2004; 72:3987-4003.
Fadl et al. Infect Immun 2005; 73:1081-1096.
Fadl et al. Infect Immun 2005; 73:8433-8436.
Hill et al. Infect. Immun. 1997; 65:4476-4482
Sing et al. J. Exp. Med. 2002; 196:1017-1024
Sing et al. Proc Natl Acad Sci USA 2005; 20
Overheim et al. Infect Immun 2005; 73:5152-5159.
Jones et al. Vaccine 2003; 21:3912-3918.
Welkos et al. Contrib. Microbiol. Immunol. 1995; 13:299-305
Welkos et al. Microb. Pathogen. 1997; 23:221-223
Worsham et al. Contrib Microbiol Immunol 1995; 13:325-328.
Welkos and O′Brien Methods Enzymol 1994; 235:29-39.
Guyton Am J Physiol 1947; 150:70-77
Byrne et al. Antimicrob Agents Chemother 1998; 42:675-681.

What is claimed is:

1. A recombinant adenovirus vector comprising a heterologous DNA segment encoding an antigenic determinant of a bioterrorism agent/disease agent classified by the United States Center for Disease Control as a category A, category B, or category C organism.

2. The vector of claim 1, wherein the organism is a fungus, a virus, or a bacteria.

3. The vector of claim 2, wherein the organism is a virus.

4. The vector of claim 3, wherein the virus is an alphavirus.

5. The vector of claim 4, wherein the alphavirus is an equine encephalitis virus.

6. The vector of claim 5, wherein the equine encephalitis virus is an eastern equine encephalitis virus (EEEV).

7. The vector of claim 6, wherein the heterologous DNA segment encodes one or more antigenic determinants comprising all or part of an E3, E2, 6K, E1 regions of EEEV, or any combination thereof.

8. The vector of claim 7, wherein the one or more antigenic determinants comprise all or part of an E3, E2, 6k, and E1 regions of EEEV.

9. The vector of claim 7, wherein the one or more antigenic determinants comprise all or part of an E3-E2 regions of EEEV.

10. The vector of claim 1, wherein the heterologous DNA segment is comprised in an expression cassette.

11. The vector of claim 2, wherein the organism is a bacteria.

12. The vector of claim 11, wherein the bacteria is Yersinia pestis.

13. The vector of claim 12, wherein the antigenic determinant comprises all or part of a Caf1, a LcrV, a YscF proteins, or any combination thereof.

14. The vector of claim 12, wherein the antigenic determinant comprises all or part of a Caf1 protein.

15. The vector of claim 12, wherein the antigenic determinant comprises all or part of a LcrV protein.

16. The vector of claim 12, wherein the antigenic determinant comprises all or part of a YscF protein.

17. The vector of claim 12, wherein the heterologous DNA segment encodes three or more antigenic determinants comprising all or part of a Caf1 protein, all or part a LcrV protein, and all or part of a YscF protein.

18. The vector of claim 1 wherein the heterologous DNA segment encodes one or more antigenic determinants of an influenza virus.

19. The vector of claim 18, wherein the influenza virus is an avian influenza virus.

20. The vector of claim 19, wherein the avian influenza virus is H5N1.

21. The vector of claim 1, wherein the heterologous DNA segment encodes one or more antigenic determinant of a toxin.

22. An isolated nucleic acid comprising an adenoviral genome comprising a heterologous DNA segment encoding an antigenic determinant of a category A, category B, or category C organism.

23. An immunogenic composition comprising an adenovirus encoding an antigenic determinant of a category A, category B, or category C organism.

24. A method of therapeutically or prophylactically immunizing a subject comprising administering a recombinant adenovirus encoding an antigenic determinant of a category A, category B, or category C organism.

25. A method for producing a protective immune response in a mammal against a biological weapon comprising administering to said mammal, a vaccine comprising an adenoviral vector having a heterologous DNA segment encoding an antigenic determinant of a category A, category B, or category C organism.

26. The method according to claim 25 wherein the mammal is either a human or a horse.

27. The method of claim 25, wherein the organism is a fungus, a virus, or a bacteria.

28. The method of claim 27, wherein the organism is a virus.

29. The method of claim 28, wherein the virus is an alphavirus.

30. The method of claim 29, wherein the alphavirus is an equine encephalitis virus.

31. The method of claim 30, wherein the equine encephalitis virus is an eastern equine encephalitis virus (EEEV).

32. The method of claim 31, wherein the heterologous DNA segment encodes one or more antigenic determinants comprising all or part of an E3, E2, 6K, E1 regions of EEEV, or any combination thereof.

33. The method of claim 31, wherein the one or more antigenic determinants comprise all or part of an E3, E2, 6k, and E1 regions of EEEV.

34. The method of claim 31, wherein the one or more antigenic determinant comprises all or part of an E3-E2 regions of EEEV.

35. The method of claim 25, wherein the heterologous DNA segment is comprised in an expression cassette.

36. The method of claim 27, wherein the organism is a bacteria.

37. The method of claim 36, wherein the bacteria is Yersinia pestis.

38. The method of claim 37, wherein the heterologous DNA segment encodes one or more antigenic determinants comprising all or part of a Caf1, a LcrV, a YscF proteins, or any combination thereof.

39. The method of claim 37, wherein the antigenic determinant comprises all or part of a Caf1 protein.

40. The method of claim 37, wherein the antigenic determinant comprises all or part of a LcrV protein.

41. The method of claim 37, wherein the antigenic determinant comprises all or part of a YscF protein.

42. The method of claim 37, wherein the one or more antigenic determinants comprise all or part of a Caf1 protein, all or part a LcrV protein, and all or part of a YscF protein.
